(19) United States
(12) Patent Application Publication
(10) Pub. No.: US 2025/0236578 A1
(43) Pub. Date: Jul. 24, 2025

(54) NOVEL CANNABINOID DERIVATIVES

(71) Applicant: CANNASOUL ANALYTICS LTD., Caesarea (IL)

(72) Inventors: Shmuel Mandel, Atlit (IL); Rotem Perry-Feigenbaum, Bazra (IL); David Meiri, Tzur Moshe (IL); Boaz Musafia, Hod-Hashron (IL); Konstantin Ulanenko, Netanya (IL); Irit Ventura, Zikhron Yaacov (IL)

(21) Appl. No.: 18/703,300

(22) PCT Filed: Nov. 10, 2022
(86) PCT No.: PCT/IL2022/051198

§ 371 Date: Apr. 19, 2024

Related U.S. Application Data
(60) Provisional application No. 63/308,089, filed on Feb. 9, 2022.

(30) Foreign Application Priority Data
Nov. 11, 2021 (IL) 288042

(51) Int. Cl. C07C 39/23; A61K 31/05; A61K 31/055; A61K 31/075; A61K 31/085; A61K 31/166; A61K 31/192; A61K 31/235; A61K 31/277; A61K 31/495; A61K 31/5375; A61K 31/7034; A61P 35/00; C07C 39/17; C07C 43/178; C07C 43/295; C07C 65/17; C07C 69/94; C07C 205/19; C07C 235/60; C07C 255/53; C07D 295/112; C07D 295/13; C07H 15/203

(52) U.S. Cl. CPC C07C 39/23; A61K 31/05; A61K 31/055; A61K 31/075; A61K 31/085; A61K 31/166; A61K 31/192; A61K 31/235; A61K 31/277; A61K 31/495; A61K 31/5375; A61K 31/7034; A61P 35/00; C07C 39/17; C07C 43/1788; C07C 43/295; C07C 65/17; C07C 69/94; C07C 205/19; C07C 235/60; C07C 255/53; C07D 295/112; C07D 295/13; C07H 15/203; C07B 2200/05; C07C 2601/14; C07C 2601/16

(57) ABSTRACT

The present invention provides novel cannabinoid derivatives, methods for their preparation, pharmaceutical compositions comprising same, and methods of use thereof as medicaments.

### FIELD OF THE INVENTION

The present invention relates to novel cannabinoid derivatives, methods for their preparation and use thereof.

### BACKGROUND OF THE INVENTION

The cannabis plant has been used as an herbal remedy for centuries. It contains more than 500 different active components including phytocannabinoids, the most prevalent ones are delta-9-tetrahydrocannabinol (A9-THC) and cannabidiol (CBD), terpenes and flavonoids. The medical use of the cannabis plant is still controversial. However, to date, there is a variety of conditions including, in particular, pain for which certain phytocannabinoids have been proven effective.

WO 2020/230145 describes a method for treating a subject afflicted with a NOTCH1-related disease comprising a step of administering to the subject a composition comprising two or more cannabinoids selected from: CF1, cannabidiol (CBD) and cannabidivarin (CBDV). NOTCH1 pathway is an evolutionarily conserved signaling system that regulates cell proliferation, differentiation, cell-fate determination and self-renewal of stem cells and progenitor cells in both embryonic and adult organs. Notably, both abnormal increases and deficiencies of NOTCH1 signaling result in human developmental anomalies and cancer development. WO 2020/230145 further describes a pharmaceutical composition comprising CF1, CBD, and CBDV.

Steroidogenesis is a multistep process for the biosynthesis of steroid hormones from cholesterol. Steroids are a large family of structurally similar lipids which function as hormonal messengers in the body regulating many organ systems including the reproductive system and the immune system. Disruption of adrenal steroidogenesis and hormone secretion may therefore affect a wide range of key processes such as the stress response, immune response, carbohydrate metabolism and many more.

Elevated steroid levels, and in particular cortisol levels, may be caused by certain medications like corticosteroids and oral contraceptives, an overactive pituitary gland, and malnutrition. Continuous high levels of cortisol may lead to the development of Cushing's syndrome, also known as hypercortisolism, which is characterized by weight gain, high blood pressure, diabetes etc.

There remains an unmet need for cannabinoid compounds having therapeutic benefits and reduced adverse effects.

### SUMMARY OF THE INVENTION

The present invention is directed to novel cannabinoid derivatives. The present invention is further directed to methods of preparing said novel cannabinoid derivatives and use thereof in the treatment of various diseases and disorders. Included within the scope of diseases and disorders are those associated with abnormal steroidogenesis and abnormal NOTCH signaling.

According to one aspect, the present invention provides a compound represented by the structure of formula I:

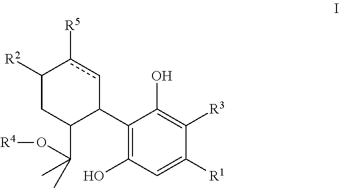

wherein
R¹ is C₁-C₁₂ alkyl, C₂-C₁₂ alkenyl, or C₂-C₁₂ alkynyl;
R² is H, deuterium, hydroxyl, halogen, haloalkyl, nitro, cyano, heterocyclyl, O-heterocyclyl, amino, or amido;
R³ is H, deuterium, hydroxyl, halogen, haloalkyl, nitro, alkoxy, aryloxy, amino, amido, or C(O)OR^{a} wherein R^{a} is H, deuterium, or C₁-C₄ alkyl;
R⁴ is H, deuterium, C₁-C₆ alkyl, C₂-C₆ alkenyl, C₂-C₆ alkynyl, aryl, or unsubstituted C(O)C₁-C₄ alkyl;
R⁵ is C₁-C₄ alkyl, C₂-C₄ alkenyl, C₂-C₄ alkynyl, or C(O)OR^{b} wherein R^{b} is H, deuterium, or C₁-C₄ alkyl; and
the dotted line represents an optional second carbon-carbon bond, wherein each of the alkyl, alkenyl, alkynyl, cyclyl, aryl, acyl, amino or amido is optionally deuterated, with the proviso that when R², R³, and R⁴ are H, R⁵ is methyl, and the dotted line represents a second carbon-carbon bond, then R¹ is not n-pentyl or 2-phenylethyl,
including salts, hydrates, solvates, polymorphs, optical isomers, geometrical isomers, enantiomers, diastereomers, and mixtures thereof. Each possibility represents a separate embodiment.

In some embodiments, the present invention provides a compound represented by the structure of formula I, wherein R¹ is C₁-C₁₂ alkyl. In other embodiments, the present invention provides a compound represented by the structure of formula I, wherein R¹ is C₁-C₁₀ alkyl. In particular embodiments, R¹ is selected from the group consisting of propyl, butyl, pentyl, hexyl, heptyl, octyl, 1,1-dimethyl pentyl, 1-methyl pentyl, 1-methyl heptyl, 1,1-dimethyl heptyl, and 1-phenyl ethyl. Each possibility represents a separate embodiment.

In certain embodiments, the present invention provides a compound represented by the structure of formula I, wherein R² is hydrogen. In other embodiments, the present invention provides a compound represented by the structure of formula I, wherein R² is halogen. In yet other embodiments, the present invention provides a compound represented by the structure of formula I, wherein R² is fluorine. In further embodiments, the present invention provides a compound represented by the structure of formula I, wherein R² is hydroxyl. In additional embodiments, the present invention provides a compound represented by the structure of formula I, wherein R² is nitro. In particular embodiments, the present invention provides a compound represented by the structure of formula I, wherein R² is cyano. In some embodiments, the present invention provides a compound represented by the structure of formula I, wherein R² is a heterocyclyl selected from the group consisting of 4-morpholinyl, and 1-piperazinyl. Each possibility represents a separate embodiment. In other embodiments, the present invention provides a compound represented by the structure of formula I, wherein R² is O-heterocyclyl, wherein the O-heterocyclyl is O-glucosyl.

In various embodiments, the present invention provides a compound represented by the structure of formula I, wherein R³ is hydrogen. In currently preferred embodiments, the present invention provides a compound represented by the structure of formula I, wherein R³ is halogen. In other currently preferred embodiments, the present invention provides a compound represented by the structure of formula I, wherein R³ is fluorine. In other embodiments, the present invention provides a compound represented by the structure of formula I, wherein R³ is hydroxyl. In additional embodiments, the present invention provides a compound represented by the structure of formula I, wherein R³ is alkoxy selected from the group consisting of methoxy, ethoxy and benzyloxy. Each possibility represents a separate embodiment. In particular embodiments, the present invention provides a compound represented by the structure of formula I, wherein R³ is C(O)OH. In additional embodiments, the present invention provides a compound represented by the structure of formula I, wherein R³ is C(O)OCH₃ or C(O)OCD₃. Each possibility represents a separate embodiment. In specific embodiments, the present invention provides a compound represented by the structure of formula I, wherein R³ is a —C(O)NHR^{c} amido, wherein R^{c} is an alkyl-amino or an alkyl-heterocyclyl. Each possibility represents a separate embodiment.

In some embodiments, the present invention provides a compound represented by the structure of formula I, wherein R⁴ is hydrogen. In other embodiments, the present invention provides a compound represented by the structure of formula I, wherein R⁴ is C₁-C₆ alkyl. In additional embodiments, the present invention provides a compound represented by the structure of formula I, wherein R⁴ is hydroxyethyl. In specific embodiments, the present invention provides a compound represented by the structure of formula I, wherein R⁴ is 2-hydroxyethyl. In yet other embodiments, the present invention provides a compound represented by the structure of formula I, wherein R⁴ is aryl. In particular embodiments, R⁴ is benzyl.

In various embodiments, the present invention provides a compound represented by the structure of formula I, wherein R⁵ is C₁-C₄ alkyl. In further embodiments, the present invention provides a compound represented by the structure of formula I, wherein R⁵ is methyl. In some embodiments, the present invention provides a compound represented by the structure of formula I, wherein R⁵ is hydroxymethyl. In additional embodiments, the present invention provides a compound represented by the structure of formula I, wherein R⁵ is C(O)OH.

In certain embodiments, the dotted line is absent. In other embodiments, the dotted line represents a second carbon-carbon bond.

In one embodiment, the present invention provides a compound represented by the structure of formula I, wherein R¹ is pentyl, R² and R⁴ are each H, R³ is halogen, R⁵ is methyl, and the dotted line represents a second carbon-carbon bond. In another embodiment, the present invention provides a compound represented by the structure of formula I, wherein R¹ is pentyl, R² and R⁴ are each H, R³ is fluorine, R⁵ is methyl, and the dotted line represents a second carbon-carbon bond.

In other embodiments, the present invention provides a compound represented by the structure of formula I, wherein R¹ is propyl, R², R³, and R⁴ are each H, R⁵ is methyl, and the dotted line represents a second carbon-carbon bond.

In further embodiments, the present invention provides a compound represented by the structure of formula I, wherein R¹ is hexyl, R², R³, and R⁴ are each H, R⁵ is methyl, and the dotted line represents a second carbon-carbon bond.

In additional embodiments, the present invention provides a compound represented by the structure of formula I, wherein R¹ is heptyl, R², R³, and R⁴ are each H, R⁵ is methyl, and the dotted line represents a second carbon-carbon bond.

In one embodiment, the present invention provides a compound represented by the structure of formula I, wherein R¹ is 1,1-dimethyl heptyl, R², R³, and R⁴ are each H, R⁵ is methyl, and the dotted line represents a second carbon-carbon bond.

In another embodiment, the present invention provides a compound represented by the structure of formula I, wherein R¹ is pentyl, R² is fluorine, R³ and R⁴ are each H, R⁵ is methyl, and the dotted line represents a second carbon-carbon bond.

In yet another embodiment, the present invention provides a compound represented by the structure of formula I, wherein R¹ is pentyl, R² and R⁴ are each H, R³ is C(O)OH, R⁵ is methyl, and the dotted line represents a second carbon-carbon bond.

In further embodiments, the present invention provides a compound represented by the structure of formula I, wherein R¹ is pentyl, R² and R⁴ are each H, R³ is C(O)OCH₃, R⁵ is methyl, and the dotted line represents a second carbon-carbon bond.

In additional embodiments, the present invention provides a compound represented by the structure of formula I, wherein R¹ is pentyl, R² and R⁴ are each H, R³ is C(O)OCD₃, R⁵ is methyl, and the dotted line represents a second carbon-carbon bond.

In other embodiments, the present invention provides a compound represented by the structure of formula I, wherein R¹ is pentyl, R², R³, and R⁴ are each H, R⁵ is methyl, and the dotted line is absent. In additional embodiments, the present invention provides a compound represented by the structure of formula I, wherein R¹ is pentyl, R² and R⁴ are each H, R³ is a —C(O)NHR^{c} amido, wherein R^{c} is -ethyldimethylamino, R⁵ is methyl, and the dotted line represents a second carbon-carbon bond.

In yet other embodiments, the present invention provides a compound represented by the structure of formula I, wherein R¹ is pentyl, R² and R⁴ are each H, R³ is a —C(O)NHR^{c} amido, wherein R^{c} is propylmorpholino, R⁵ is methyl, and the dotted line represents a second carbon-carbon bond.

It is to be understood for all compounds of the present invention encompassed by the structure of formula I, that when R², R³, and R⁴ are H, R⁵ is methyl, and the dotted line represents a second carbon-carbon bond, then R¹ is not a straight-chain, unsubstituted C₅H₁₁ alkyl, i.e., n-pentyl. It is further to be understood for all compounds of the present invention encompassed by the structure of formula I, that when R², R³, and R⁴ are H, R⁵ is methyl, and the dotted line represents a second carbon-carbon bond, then R¹ is not 2-phenylethyl, i.e., 2-phenyl-1-ethyl.

Representative and non-limiting examples of such structures are compounds selected from the group consisting of compounds 1-37:

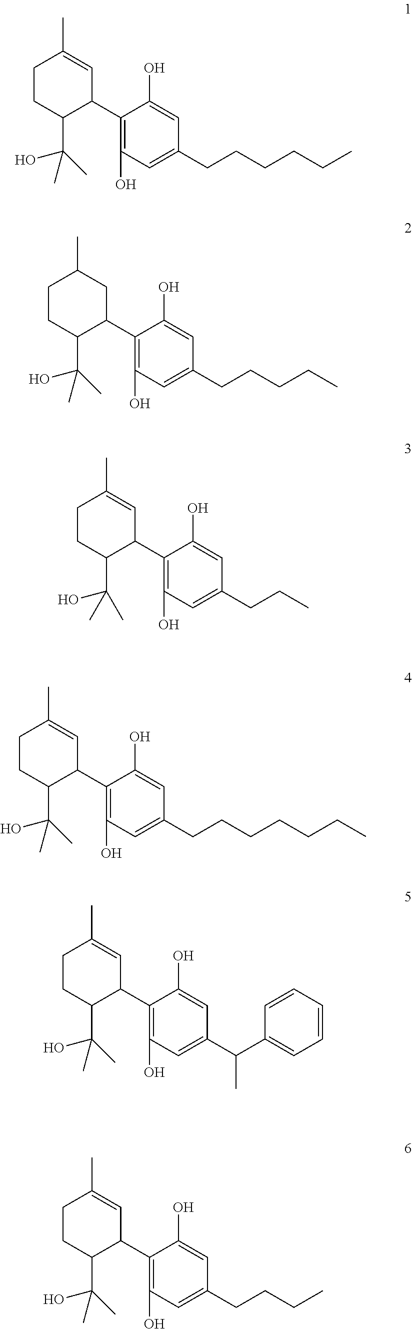 [SMILES: CCCCCCc1cc(O)c(C2C=C(C)CCC2C(C)(C)O)c(O)c1 ; CCCCCc1cc(O)c(C2CC(C)CCC2C(C)(C)O)c(O)c1 ; CCCc1cc(O)c(C2C=C(C)CCC2C(C)(C)O)c(O)c1 ; CCCCCCCc1cc(O)c(C2C=C(C)CCC2C(C)(C)O)c(O)c1 ; CC1=CC(c2c(O)cc(C(C)c3ccccc3)cc2O)C(C(C)(C)O)CC1 ; CCCCc1cc(O)c(C2C=C(C)CCC2C(C)(C)O)c(O)c1]

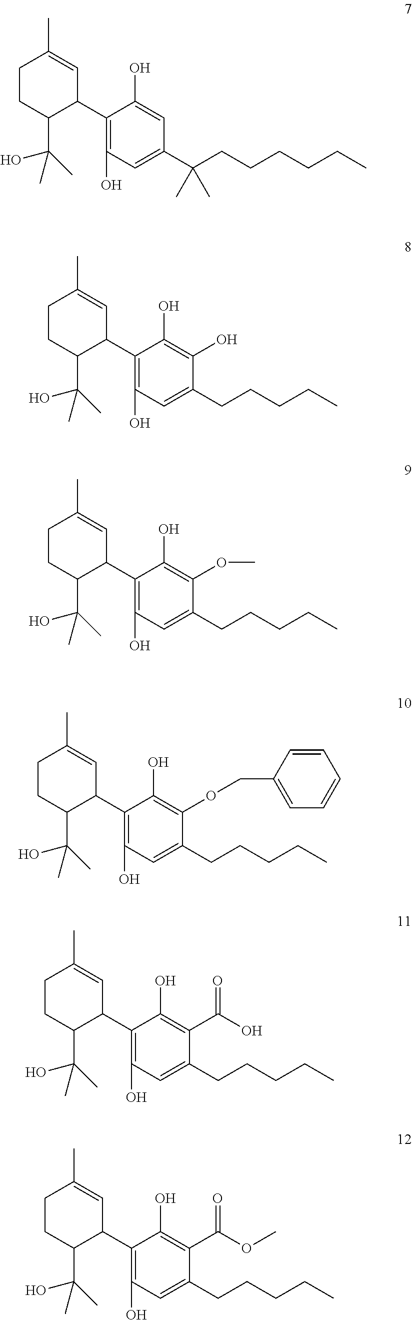 [SMILES: CCCCCCC(C)(C)c1cc(O)c(C2C=C(C)CCC2C(C)(C)O)c(O)c1 ; CCCCCc1cc(O)c(C2C=C(C)CCC2C(C)(C)O)c(O)c1O ; CCCCCc1cc(O)c(C2C=C(C)CCC2C(C)(C)O)c(O)c1OC ; CCCCCc1cc(O)c(C2C=C(C)CCC2C(C)(C)O)c(O)c1OCc1ccccc1 ; CCCCCc1cc(O)c(C2C=C(C)CCC2C(C)(C)O)c(O)c1C(=O)O ; CCCCCc1cc(O)c(C2C=C(C)CCC2C(C)(C)O)c(O)c1C(=O)OC]

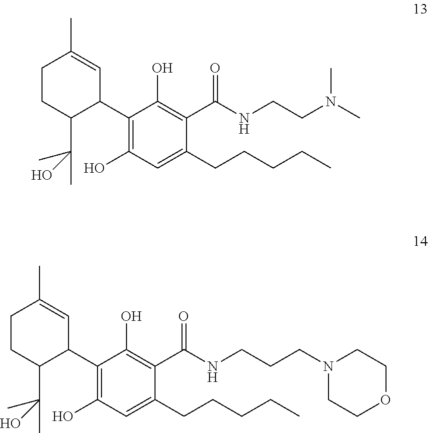

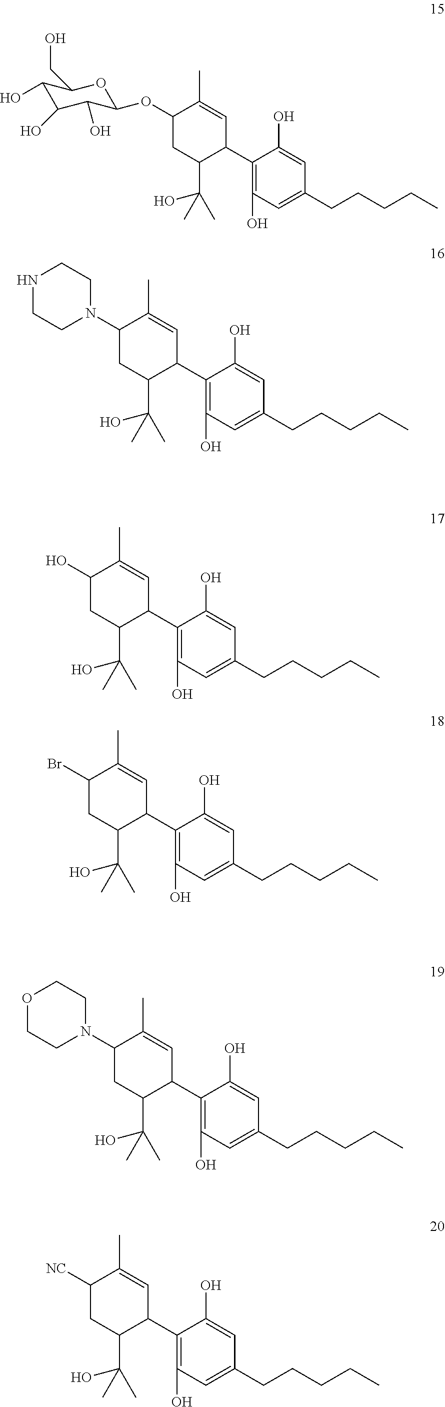

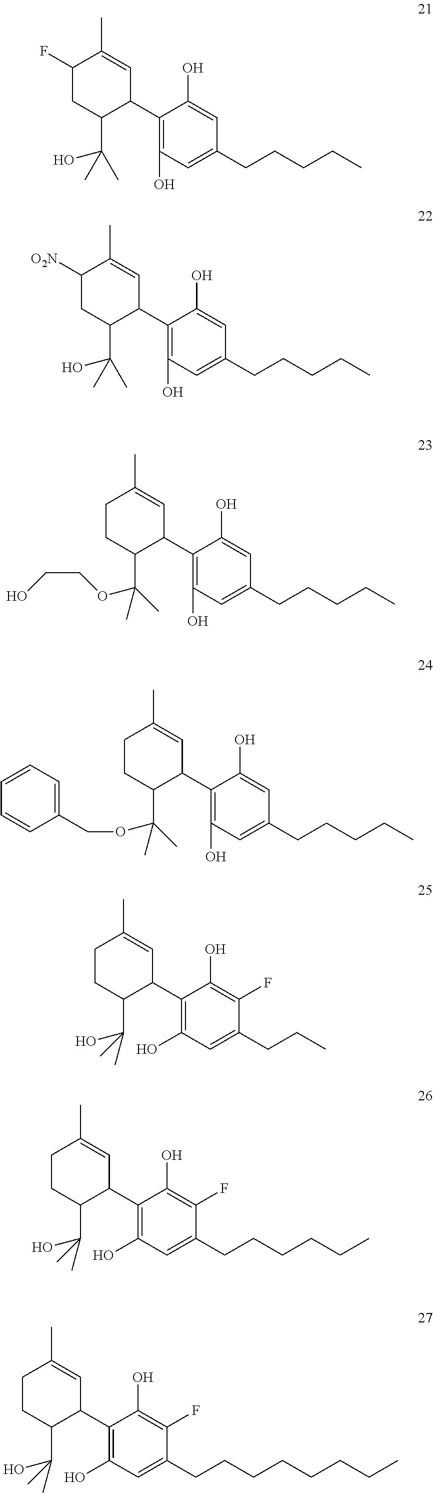

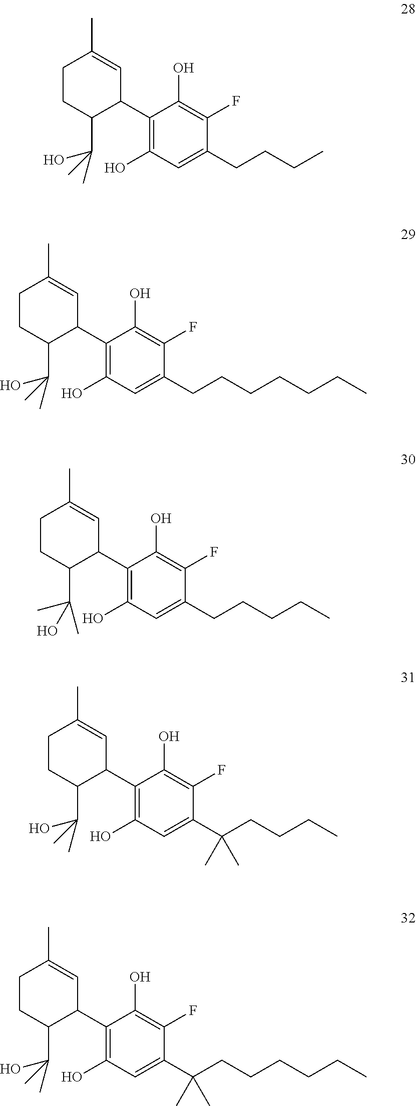

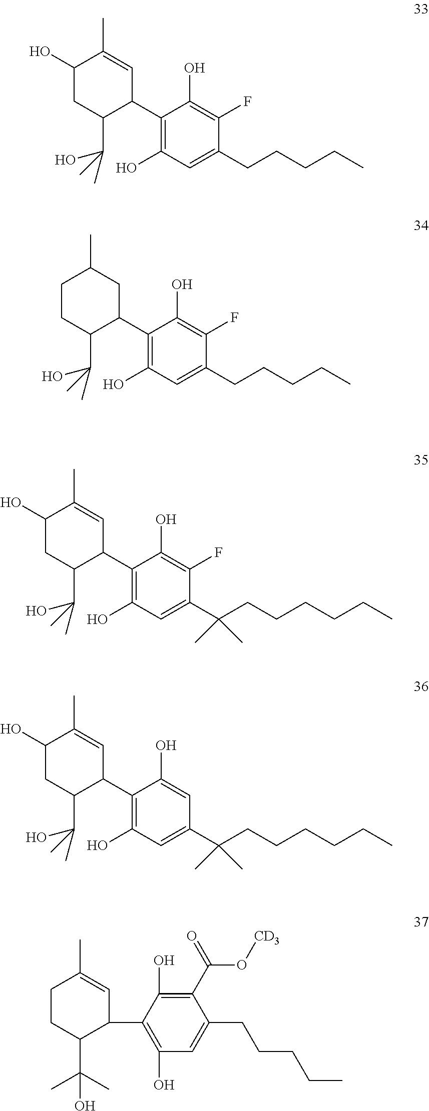 [SMILES: CCCCCc1cc(O)c(C2C=C(C)C(O)CC2C(C)(C)O)c(O)c1F ; CCCCCc1cc(O)c(C2CC(C)CCC2C(C)(C)O)c(O)c1F ; CCCCCCC(C)(C)c1cc(O)c(C2C=C(C)C(O)CC2C(C)(C)O)c(O)c1F ; CCCCCCC(C)(C)c1cc(O)c(C2C=C(C)C(O)CC2C(C)(C)O)c(O)c1 ; [2H]C([2H])([2H])OC(=O)c1c(CCCCC)cc(O)c(C2C=C(C)CCC2C(C)(C)O)c1O]

including salts, hydrates, solvates, polymorphs, optical isomers, geometrical isomers, enantiomers, diastereomers, and mixtures thereof. Each possibility represents a separate embodiment.

The present invention further provides a pharmaceutical composition comprising a therapeutically effective amount of at least one compound represented by the structure of formula I and a pharmaceutically acceptable carrier or excipient. In one embodiment, the pharmaceutical composition comprises the compound disclosed herein as the sole active ingredient. In another embodiment, the pharmaceutical composition comprises the compound represented by the structure of formula I in combination with one or more cannabinoids, terpenes, terpenoids, flavonoids, oils, nitrogenous compounds, amino acids, proteins, glycoproteins, sugars, hydrocarbons, fatty acids, esters, lactones, steroids, non-cannabinoid phenols, and a mixture or combination thereof. Each possibility represents a separate embodiment.

In some embodiments, the pharmaceutically acceptable carrier or excipient comprises at least one of a binder, a filler, a diluent, a surfactant or emulsifier, a glidant or lubricant, a buffering or pH adjusting agent, a tonicity enhancing agent, a wetting agent, a chelating agent, a preservative, an antioxidant, a flavoring agent, a colorant, and a mixture or combination thereof. Each possibility represents a separate embodiment. In one particular embodiment, the pharmaceutically acceptable carrier is a lipid carrier.

In other embodiments, the pharmaceutical composition is in a form selected from the group consisting of tablet, pill, capsule (e.g., soft or hard gelatin capsule), pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, ointment, suppository, a gel, and a cream. Each possibility represents a separate embodiment.

In further embodiments, the pharmaceutical composition is formulated (or adapted) for administration via a route selected from the group consisting of oral, topical, transdermal, intra-arterial, intranasal, intraperitoneal, intramuscular, subcutaneous, intravenous, and intra-alveolar. Each possibility represents a separate embodiment.

In some embodiments, the present invention provides a pharmaceutical composition comprising a therapeutically effective amount of at least one compound represented by the structure of formula I and a pharmaceutically acceptable carrier or excipient for use as a medicament.

In particular embodiments, the present invention provides a pharmaceutical composition comprising a therapeutically effective amount of at least one compound represented by the structure of formula I and a pharmaceutically acceptable carrier or excipient for use in treating a disease or disorder characterized by overproduction of a steroid.

In further embodiments, the present invention provides a method of treating a disease or disorder characterized by overproduction of a steroid in a subject in need thereof, the method comprising administering to the subject a pharmaceutical composition comprising a therapeutically effective amount of at least one compound represented by the structure of formula I and a pharmaceutically acceptable carrier or excipient.

In specific embodiments, the disease or disorder is selected from the group consisting of neurodegenerative disorders, metabolic disorders, psychiatric disorders, and trauma-related disorders. Each possibility represents a separate embodiment.

In particular embodiments, the neurodegenerative disorder is selected from the group consisting of Alzheimer's disease, Parkinson's disease, Huntington's disease, amyotrophic lateral sclerosis, Machado-Joseph disease, Creutzfeldt-Jakob disease, and age-related macular degeneration. Each possibility represents a separate embodiment.

In other embodiments, the metabolic disorder is selected from the group consisting of type 1 diabetes mellitus, type 2 diabetes mellitus, impaired glucose tolerance, hyperglycemia, overweight, obesity, weight gain, hypertension, hypercholesterolemia, hyperlipidemia, hypertriglyceridemia, fatty liver, coronary heart disease, cardiovascular disease, hyperpituitarism, and Cushing's syndrome. Each possibility represents a separate embodiment.

In yet other embodiments, the psychiatric disorder is selected from the group consisting of depressive disorder including post-partum depression, anxiety disorder, eating disorders including anorexia nervosa and bulimia, alcoholism or alcohol abuse, drug abuse, sleep disorder, premenstrual dysphoric disorder, mood disorder, aggressiveness, convulsions, stress disorder including posttraumatic stress disorder, schizophrenia, chronic fatigue syndrome, obsessive compulsive disorder, panic disorder, pre-menstrual syndrome (PMS), phobia including social phobia and agoraphobia, mania, manic-depression (bipolar disorder), smoking cessation and nicotine withdrawal syndrome. Each possibility represents a separate embodiment.

In further embodiments, the trauma-related disorder is selected from the group consisting of brain injury, ischemia-reperfusion injury, and stroke including ischemic stroke. Each possibility represents a separate embodiment.

In other embodiments, the present invention provides a pharmaceutical composition comprising a therapeutically effective amount of at least one compound represented by the structure of formula I and a pharmaceutically acceptable carrier or excipient for use in treating a proliferative disease or disorder.

In additional embodiments, the present invention provides a method of treating a proliferative disease or disorder in a subject in need thereof, the method comprising administering to the subject a pharmaceutical composition comprising a therapeutically effective amount of at least one compound represented by the structure of formula I and a pharmaceutically acceptable carrier or excipient.

In specific embodiments, the proliferative disease or disorder is cancer.

In various embodiments, the cancer is selected from the group consisting of head and neck cancer, sarcoma, multiple myeloma, ovarian cancer, breast cancer, bladder cancer, kidney cancer, stomach cancer, hematopoietic cancers, lymphoma, leukemia, lung carcinoma, melanoma, glioblastoma, hepatocarcinoma, prostate cancer, pancreatic cancer, and colon cancer. Each possibility represents a separate embodiment.

In particular embodiments, the cancer is selected from the group consisting of T cell acute lymphoblastic leukemia (T-ALL), chronic lymphocytic leukemia (CLL), melanoma, cholangiocarcinoma (CCC), colorectal cancer, lung adenocarcinoma, glioblastoma, renal cell carcinoma, ovarian cancer, prostate cancer, breast cancer, pancreatic ductal adenocarcinoma (PDAC), cervical cancer, head and neck squamous cell carcinomas (HNSCC), hepatocellular carcinoma (HCC), medulloblastoma, B cell acute lymphoblastic leukemia (B-ALL), acute myeloid leukemia (AML), small cell lung carcinoma (SCLC), non-small-cell lung carcinoma (NSCLC), lung squamous cell carcinoma (SqCC), cutaneous squamous cell carcinoma (SqCC), chronic myelomonocytic leukemia (CMML), cutaneous T-cell lymphoma (CTCL) including mycosis fungoides and Sézary syndrome, intestinal and diffuse-type gastric cancer, pilocytic astrocytoma (PA), choroid plexus tumor, laryngeal squamous cell carcinoma (LSCC), gallbladder carcinoma, Kaposi's sarcoma, B cell malignancy, T cell lymphoma, pancreatic cancer, nasopharyngeal carcinoma, squamous cell carcinoma, prostatic adenocarcinoma, infantile myofibromatosis (IM), lateral meningocele syndrome, and desmoid tumor. Each possibility represents a separate embodiment.

In other embodiments, the treatment of cancer comprises modulating the expression of a NOTCH protein. In yet other embodiments, the treatment of cancer comprises modulating the abnormal expression of a NOTCH protein. In further embodiments, the treatment of cancer comprises modulating the abnormal expression of a NOTCH protein which is encoded by a NOTCH gene comprising at least one mutation.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention relates to novel cannabinoids and methods for their preparation.

The novel cannabinoids of the present invention are designed to treat various diseases and disorders including, but not limited to, neurodegenerative disorders, metabolic disorders, psychiatric disorders, trauma-related disorders, and proliferative disorders.

### Cannabinoid Derivatives

The present invention provides compounds that are represented by the structure of formula I:

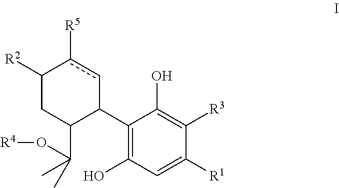

wherein
R¹ is C₁-C₁₂ alkyl, C₂-C₁₂ alkenyl, or C₂-C₁₂ alkynyl;
R² is H, deuterium, hydroxyl, halogen, haloalkyl, nitro, cyano, heterocyclyl, O-heterocyclyl, amino, or amido;
R³ is H, deuterium, hydroxyl, halogen, haloalkyl, nitro, alkoxy, aryloxy, amino, amido, or C(O)OR^{a} wherein R^{a} is H, deuterium, or C₁-C₄ alkyl;
R⁴ is H, deuterium, C₁-C₆ alkyl, C₂-C₆ alkenyl, C₂-C₆ alkynyl, aryl, or acyl;
R⁵ is C₁-C₄ alkyl, C₂-C₄ alkenyl, C₂-C₄ alkynyl, or C(O)OR^{b} wherein R^{b} is H, deuterium, or C₁-C₄ alkyl; and
the dotted line represents an optional second carbon-carbon bond, wherein each of the alkyl, alkenyl, alkynyl, cyclyl, aryl, acyl, amino or amido is optionally deuterated, with the proviso that when R², R³, and R⁴ are H, R⁵ is methyl, and the dotted line represents a second carbon-carbon bond, then R¹ is not n-pentyl or 2-phenylethyl,
including salts, hydrates, solvates, polymorphs, optical isomers, geometrical isomers, enantiomers, diastereomers, and mixtures thereof. Each possibility represents a separate embodiment.

The present invention further provides currently preferred embodiments wherein formula I comprises the following substitutions with the proviso that when R², R³, and R⁴ are H, R⁵ is methyl, and the dotted line represents a second carbon-carbon bond, then R¹ is not n-pentyl or 2-phenylethyl:
1. R¹ is C₁-C₁₂ alkyl, preferably C₁-C₁₀ alkyl.
2. R¹ is selected from the group consisting of propyl, butyl, pentyl, hexyl, heptyl, octyl, 1,1-dimethyl pentyl, 1-methyl pentyl, 1-methyl heptyl, 1,1-dimethyl heptyl, and 1-phenyl ethyl. Each possibility represents a separate embodiment.
3. R² is hydrogen.
4. R² is halogen, preferably fluorine.
5. R² is hydroxyl.
6. R² is nitro.
7. R² is cyano.
8. R² is a heterocyclyl selected from the group consisting of 4-morpholinyl, and 1-piperazinyl. Each possibility represents a separate embodiment.
9. R² is O-heterocyclyl, preferably O-glucosyl.
10. R³ is hydrogen.
11. R³ is halogen, preferably fluorine.
12. R³ is hydroxyl.
13. R³ is an alkoxy selected from the group consisting of methoxy, ethoxy and benzyloxy. Each possibility represents a separate embodiment.
14. R³ is C(O)OH.
15. R³ is C(O)OCH₃ or C(O)OCD₃. Each possibility represents a separate embodiment.
16. R³ is a —C(O)NHR^{c} amido, wherein R^{c} is an alkyl-amino or an alkyl-heterocyclyl. Each possibility represents a separate embodiment.
17. R⁴ is hydrogen.
18. R⁴ is C₁-C₆ alkyl.
19. R⁴ is hydroxyethyl, preferably 2-hydroxyethyl.
20. R⁴ is aryl, preferably benzyl.
21. R⁵ is C₁-C₄ alkyl.
22. R⁵ is methyl.
23. R⁵ is CH₂OH.
24. R⁵ is C(O)OH.

According to certain aspects and embodiments, the dotted line is absent and the ring is a cyclohexyl ring. According to other aspects and embodiments, the dotted line represents a second carbon-carbon bond and the ring is a cyclohexenyl ring.

According to various aspects and embodiments, R¹ is pentyl, R² and R⁴ are each H, R³ is a halogen, R⁵ is methyl, and the dotted line represents a second carbon-carbon bond. In other aspects and embodiments, R¹ is pentyl, R² and R⁴ are each H, R³ is fluorine, R⁵ is methyl, and the dotted line represents a second carbon-carbon bond.

In other aspects and embodiments, R¹ is propyl, R², R³ and R⁴ are each H, R⁵ is methyl, and the dotted line represents a second carbon-carbon bond.

In yet other aspects and embodiments, R¹ is hexyl, R², R³, and R⁴ are each H, R⁵ is methyl, and the dotted line represents a second carbon-carbon bond.

In additional aspects and embodiments, R¹ is heptyl, R², R³, and R⁴ are each H, R⁵ is methyl, and the dotted line represents a second carbon-carbon bond.

In other embodiments, R¹ is 1,1-dimethyl heptyl, R², R³, and R⁴ are each H, R⁵ is methyl, and the dotted line represents a second carbon-carbon bond.

In further aspects and embodiments, R¹ is pentyl, R² is fluorine, R³ and R⁴ are each H, R⁵ is methyl, and the dotted line represents a second carbon-carbon bond.

In additional aspects and embodiments, R¹ is pentyl, R² and R⁴ are each H, R³ is C(O)OH, C(O)OCH₃, or C(O)OCD₃, R⁵ is methyl, and the dotted line represents a second carbon-carbon bond. Each possibility represents a separate embodiment.

In some aspects and embodiments, R¹ is pentyl, R², R³, and R⁴ are each H, R⁵ is methyl, and the dotted line is absent.

In particular aspects and embodiments, R¹ is pentyl, R² and R⁴ are each H, R³ is a —C(O)NHR^{c} amido, wherein R^{c} is -ethyldimethylamino, R⁵ is methyl, and the dotted line represents a second carbon-carbon bond.

In other aspects and embodiments, R¹ is pentyl, R² and R⁴ are each H, R³ is a —C(O)NHR^{c} amido, wherein R^{c} is -propylmorpholino, R⁵ is methyl, and the dotted line represents a second carbon-carbon bond.

Representative and non-limiting examples of such structures are compounds selected from the group consisting of compounds 1 to 37, with each possibility representing a separate embodiment.

### Chemical Definitions

An “alkyl” group refers to any unsubstituted or substituted alkyl. An “unsubstituted alkyl” group refers to a saturated aliphatic hydrocarbon, including straight-chain, branched-chain and cyclic alkyl groups. In one embodiment, the alkyl group has 1-12 carbons designated herein as C₁-C₁₂ alkyl. In another embodiment, the alkyl group has 1-6 carbons designated herein as C₁-C₆ alkyl. In yet another embodiment, the alkyl group has 1-4 carbons designated here in as C₁-C₄ alkyl. A “substituted alkyl” group refers to an alkyl which is substituted by one or more groups selected from halogen, hydroxyl, aryl, heterocyclyl, amino and amido. Each possibility represents a separate embodiment. Non-limiting examples of alkyl substituents include benzyl, 1-phenylethyl, 2-(dimethylamino)-ethyl, 3-(morpholino)-propyl, and 2-hydroxyethyl. Each possibility represents a separate embodiment.

An “alkenyl” group refers to an aliphatic hydrocarbon group containing at least one carbon-carbon double bond including straight-chain, branched-chain and cyclic alkenyl groups. In one embodiment, the alkenyl group has 2-12 carbon atoms designated here as C₂-C₁₂ alkenyl. In another embodiment, the alkenyl group has 2-6 carbon atoms in the chain designated here as C₂-C₆ alkenyl. Exemplary alkenyl groups include ethenyl, propenyl, n-butenyl, i-butenyl, 3-methylbut-2-enyl, n-pentenyl, heptenyl, octenyl, cyclohexyl-butenyl and decenyl. Each possibility represents a separate embodiment. The alkenyl group can be unsubstituted or substituted through available carbon atoms with one or more groups defined hereinabove for alkyl.

An “alkynyl” group refers to an aliphatic hydrocarbon group containing at least one carbon-carbon triple bond including straight-chain and branched-chain. In one embodiment, the alkynyl group has 2-12 carbon atoms in the chain designated here as C₂-C₁₂ alkynyl. In another embodiment, the alkynyl group has 2-6 carbon atoms in the chain designated here as C₂-C₆ alkynyl. Exemplary alkynyl groups include ethynyl, propynyl, n-butynyl, 2-butynyl, 3-methylbutynyl, n-pentynyl, heptynyl, octynyl and decynyl. Each possibility represents a separate embodiment. The alkynyl group can be unsubstituted or substituted through available carbon atoms with one or more groups defined hereinabove for alkyl.

The term “aryl” used herein alone or as part of another group refers to an aromatic ring system containing from 6 to 14 ring carbon atoms. The aryl ring can be a monocyclic, bicyclic, tricyclic and the like. Non-limiting examples of aryl groups are phenyl, naphthyl including 1-naphthyl and 2-naphthyl, and the like. Each possibility represents a separate embodiment. The aryl group can be unsubstituted or substituted through available carbon atoms with one or more groups defined hereinabove for alkyl.

The term “heterocyclic ring” or “heterocyclyl” used herein alone or as part of another group refers to five-membered to eight-membered rings that have 1 to 4 heteroatoms, such as oxygen, sulfur and/or nitrogen, in particular oxygen, as a ring atom. These five-membered to eight-membered rings can be saturated, fully unsaturated or partially unsaturated, with fully saturated rings being preferred. Exemplary heterocyclic rings include piperidinyl, pyrrolidinyl pyrrolinyl, pyrazolinyl, pyrazolidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, dihydrothiazolyl, glucosyl, and the like. Each possibility represents a separate embodiment. The heterocyclyl group can be unsubstituted or substituted through available atoms with one or more groups defined hereinabove for alkyl. It is to be understood that “O-heterocyclyl” includes an oxygen linked to a heterocyclyl group as defined hereinabove. An exemplary O-heterocyclyl includes, but is not limited to, O-glycosyl.

The term “acyl” as used herein encompasses groups such as, but not limited to, formyl, acetyl, propionyl, butyryl, pentanoyl, pivaloyl, hexanoyl, heptanoyl, octanoyl, nonanoyl, decanoyl, undecanoyl, dodecanoyl, benzoyl and the like. Each possibility represents a separate embodiment. In one embodiment, the acyl is an unsubstituted C(O)C₁-C₄ alkyl. In another embodiment, the acyl is other than C(O)CF₃.

A “hydroxy” group refers to an —OH group.

An “alkoxy” group refers to an —O-alkyl group wherein alkyl is as defined above. Exemplary alkoxy groups include, but are not limited to, methoxy, ethoxy, benzyloxy, and the like. Each possibility represents a separate embodiment.

An “aryloxy” group refers to an —O-aryl group wherein aryl is as defined above. Exemplary aryloxy groups include, but are not limited to, phenyloxy, and the like.

An “amino” group refers to an —NH₂ group, an —NHR group, or an —NRR′ group, wherein R and R′ are each independently alkyl, alkenyl, alkynyl, aryl, heterocyclyl, alkyl-amino, or alkyl-heterocyclyl as defined above. Each possibility represents a separate embodiment.

An “amido” group refers to a —C(O)NH₂ group, —C(O)NHR group, or a —C(O)NRR′ group wherein R and R′ are each independently unsubstituted or substituted alkyl (including alkyl-amino, or alkyl-heterocyclyl), alkenyl, alkynyl, aryl, or heterocyclyl, as defined above. Each possibility represents a separate embodiment.

A “halogen” refers to chlorine, bromine, fluorine, and iodine. Each possibility represents a separate embodiment.

A “haloalkyl” refers to an alkyl group having some or all of the hydrogens independently replaced by a halogen. Exemplary haloalkyls include, but are not limited to, trichloromethyl, tribromomethyl, trifluoromethyl, triiodomethyl, difluoromethyl, chlorodifluoromethyl, pentafluoroethyl, 1,1-difluoroethyl bromomethyl, chloromethyl, fluoromethyl, iodomethyl, and the like. Each possibility represents a separate embodiment.

A “nitro” group refers to an —NO₂ group.

A “cyano” group refers to a —CN group.

As used herein, the term “optionally deuterated” refers to a group that can be unsubstituted or substituted with at least one deuterium, i.e., one or more hydrogens can be replaced by one or more deuterons. In some embodiments, all hydrogens in the group are replaced by deuterons.

All stereoisomers of the compounds of the present invention are contemplated, either in admixture or in pure or substantially pure form. These compounds can have asymmetric centers at one or more atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. Where compounds of the invention contain a double bond (for example where R¹ is a C₂-C₁₂ alkenyl), it is to be understood that it encompasses all structural and geometrical isomers including cis, trans, E and Z isomers and optical isomers, independently at each occurrence.

One or more of the compounds of the invention, may be present as a salt. The term “salt” as used herein encompasses both base and acid addition salts including, but not limited to, carboxylate salts or salts with amine nitrogens, and include salts formed with the organic and inorganic anions and cations detailed below. Further encompassed by the term are salts formed by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and the like. Each possibility represents a separate embodiment.

The term “organic or inorganic cation” refers to counter-ions for the carboxylate anion of a carboxylate salt. The counter-ions are chosen from the alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. Each possibility represents a separate embodiment. Other cations encompassed by the above term include the protonated form of procaine, quinine and N-methylglucosamine. Each possibility represents a separate embodiment. Furthermore, any zwitterionic form of the instant compounds formed by a carboxylic acid and an amino group are also contemplated.

The present invention also includes solvates of any of compounds represented by formula I or any of compounds 1-37 and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of any of compounds represented by formula I or any of compounds 1-37 and salts thereof. The term “polymorph” refers to a particular crystalline state of a substance, which can be characterized by particular physical properties such as X-ray diffraction, IR spectra, melting point, and the like.

### Pharmaceutical Compositions

According to certain aspects and embodiments, the invention relates to a pharmaceutical composition comprising a therapeutically effective amount of a compound of formulae I or 1-37 including salts, hydrates, solvates, polymorphs, optical isomers, geometrical isomers, enantiomers, diastereomers, and mixtures thereof, the pharmaceutical composition further comprising a pharmaceutically acceptable carrier or excipient.

Suitable pharmaceutically acceptable carriers or excipients include, but are not limited to, a binder, a filler, a diluent, a surfactant or emulsifier, a glidant or lubricant, buffering or pH adjusting agent, a tonicity enhancing agent, a wetting agent, a chelating agent, a preservative, an antioxidant, a flavoring agent, a colorant, and a mixture or combination thereof. Each possibility represents a separate embodiment.

Suitable binders include, but are not limited to, polyvinylpyrrolidone, copovidone, hydroxypropyl methylcellulose, starch, and gelatin. Each possibility represents a separate embodiment.

Suitable fillers include, but are not limited to, sugars such as lactose, sucrose, mannitol or sorbitol and derivatives therefore (e.g. amino sugars), ethylcellulose, microcrystalline cellulose, and silicified microcrystalline cellulose. Each possibility represents a separate embodiment.

Suitable lubricants include, but are not limited to, sodium stearyl fumarate, stearic acid, polyethylene glycol or stearates, such as magnesium stearate. Each possibility represents a separate embodiment.

Suitable diluents include, but are not limited to, dicalcium phosphate dihydrate, sugars, lactose, calcium phosphate, cellulose, kaolin, mannitol, sodium chloride, starch, and various oils. Each possibility represents a separate embodiment.

Suitable surfactants or emulsifiers include, but are not limited to, polyvinyl alcohol (PVA), polysorbate, polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil and hardened castor oil such as polyoxyethylene hardened castor oil, and lecithin. Each possibility represents a separate embodiment.

Suitable glidants or lubricants include, but are not limited to, colloidal silicon dioxide, magnesium stearate, talc, and mineral oil. Each possibility represents a separate embodiment.

Suitable buffering or pH adjusting agents include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide. Each possibility represents a separate embodiment.

Suitable tonicity enhancing agents include, but are not limited to, ionic and non-ionic agents such as, alkali metal or alkaline earth metal halides, urea, glycerol, sorbitol, mannitol, propylene glycol, and dextrose. Each possibility represents a separate embodiment.

Suitable wetting agents include, but are not limited to, glycerin, cetyl alcohol, and glycerol monostearate. Each possibility represents a separate embodiment.

Suitable chelating agents include, but are not limited to, modified or unmodified cyclodextrin (e.g. α-cyclodextrin, β-cyclodextrin, γ-cyclodextrin, 2-hydroxypropyl-β-cyclodextrin, methyl-β-cyclodextrin), dextrin, maltodextrin, and a mixture or combination thereof. Each possibility represents a separate embodiment.

Suitable preservatives include, but are not limited to, benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide. Each possibility represents a separate embodiment.

Suitable antioxidants include, but are not limited to, sorbic acid, ascorbic acid, ascorbate, glycine, α-tocopherol, butylated hydroxyanisole (BHA), and butylated hydroxytoluene (BHT). Each possibility represents a separate embodiment.

Suitable flavoring agents include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot. Each possibility represents a separate embodiment.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide. Each possibility represents a separate embodiment.

In certain aspects and embodiments, the pharmaceutical composition of the present invention is formulated as tablet, pill, capsule (e.g. soft or hard gelatin capsule), pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, ointment, suppository, a gel, and a cream. Each possibility represents a separate embodiment.

For preparing solid compositions such as tablets, the active pharmaceutical ingredient is mixed with a pharmaceutical carrier or excipient to form a solid pre-formulation composition containing a substantially homogeneous distribution of the compound of the present invention in the pharmaceutical carrier or excipient.

Any method can be used to prepare the pharmaceutical compositions. For example, solid dosage forms can be prepared by wet granulation, dry granulation, direct compression and the like as is known in the art. The liquid forms in which the compounds of the present invention may be incorporated, for administration via a route selected from oral, topical or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Each possibility represents a separate embodiment.

The compositions of the present invention may be formulated as a single-phase aqueous emulsion or multiple emulsions. According to some embodiments, the composition is formulated as an emulsion. These emulsions may be oil-in-water (o/w) emulsions, water-in-oil (w/o) emulsions, or multiple emulsions such as oil-in-water-in-oil (o/w/o) or water-in-oil-in-water (w/o/w) double emulsions. It is understood that the oil phase can comprise silicone oils, non-silicone organic oils, or mixtures thereof. The compositions can comprise two immiscible phases that are reconstituted prior to use. Each possibility represents a separate embodiment of the present invention.

In certain embodiments, the compositions of the present invention are liposomal compositions comprising a compound represented by the structure of formula I or any of compounds 1-37 as defined herein encapsulated in a liposome comprising a lipid bilayer structure.

Another formulation encompassed by the present invention comprises transdermal delivery devices (“patches”). Such transdermal patches may be used to provide continuous or discontinuous infusion of the compound of the present invention in controlled amounts. The construction and use of transdermal patches for the delivery of pharmaceutical agents is well known in the art.

In yet another embodiment, the composition is prepared for topical administration, e.g. as an oil, ointment, gel or cream. Adjuvants for topical administration may include, for example, sodium carboxymethylcellulose, polyacrylates, polyoxyethylene-polyoxypropylene-block polymers, polyethylene glycol and wood wax alcohols. The term “gel” as used herein, refers to a substantially dilute cross-linked system, which exhibits little or no flow when in the steady-state having a solid jelly-like matrix. As contemplated herein, gel may comprise hydrogel, organogel, thermosensitive gel, non-thermosensitive gel, and aerogel. Each possibility represents a separate embodiment.

Compositions for inhalation or aspiration include solutions and suspensions in pharmaceutically acceptable aqueous or organic solvents, or mixtures thereof, as well as powders. The liquid or solid compositions may contain suitable pharmaceutically acceptable carriers or excipients as described above. The compositions may be administered by the oral or nasal respiratory route. Compositions may also be nebulized by use of inert gases. Nebulized solutions may be breathed directly from the nebulizing device or the nebulizing device may be attached to a face mask tent, or intermittent positive pressure breathing machine. Solution, suspension, or powder compositions may be administered, preferably orally or nasally, from devices (e.g. inhalers) that deliver the formulation in an appropriate manner.

The pharmaceutical compositions of the present invention may exhibit release mode which may be immediate release, controlled release or a mixture thereof. Each possibility represents a separate embodiment of the invention. “Immediate release” (IR) compositions in the context of the present invention refers to compositions in which the active ingredient is released without delay following administration. “Controlled release” (CR) compositions in the context of the present invention refers to compositions in which the active ingredient is released gradually over a period of time following administration.

### Therapeutic Uses

The pharmaceutical composition comprising a therapeutically effective amount of at least one compound according to the present invention and a pharmaceutically acceptable carrier or excipient can be used as a medicament. Thus, there is provided a pharmaceutical composition comprising a therapeutically effective amount of a compound of formulae I or 1-37 including salts, hydrates, solvates, polymorphs, optical isomers, geometrical isomers, enantiomers, diastereomers, and mixtures thereof; and further comprising a pharmaceutically acceptable carrier or excipient for use as a medicament.

In some aspects and embodiments, the compounds and pharmaceutical compositions comprising same are useful for treating neurodegenerative disorders, metabolic disorders, psychiatric disorders, trauma-related disorders, and proliferative disorders. Each possibility represents a separate embodiment.

The term “neurodegenerative disorders” refers to diseases in which the function of a subject's nervous system becomes impaired. Examples of neurodegenerative diseases that may be treated with the compound or composition described herein include, but are not limited to, Alzheimer's disease, Parkinson's disease, Huntington's disease, amyotrophic lateral sclerosis, Machado-Joseph disease, Creutzfeldt-Jakob disease, and age-related macular degeneration. Each possibility represents a separate embodiment.

The compounds and compositions of the present invention can be utilized for treating metabolic disorders.

The term “metabolic disorders” refers to diseases associated with lipid metabolism. In particular, the term “metabolic disorder” as used herein refers a condition which is characterized by an alteration or imbalance in metabolic function. Examples of metabolic disorders which can be treated by the compounds and compositions of the present invention include, but are not limited to, type 1 diabetes mellitus, type 2 diabetes mellitus, impaired glucose tolerance, hyperglycemia, overweight, obesity, weight gain, hypertension, hypercholesterolemia, hyperlipidemia, hypertriglyceridemia, coronary heart disease, cardiovascular disease, hyperpituitarism, and Cushing's syndrome. Each possibility represents a separate embodiment.

The term “psychiatric disorders” refers to neurological diseases comprising mental diseases. Examples of psychiatric disorders that may be treated with the compound or composition described herein include, but are not limited to, depressive disorder including post-partum depression, anxiety disorder, eating disorders including anorexia nervosa and bulimia, alcoholism or alcohol abuse, drug abuse, sleep disorder, premenstrual dysphoric disorder, mood disorder, aggressiveness, convulsions, stress disorder including posttraumatic stress disorder, schizophrenia, chronic fatigue syndrome, obsessive compulsive disorder, panic disorder, pre-menstrual syndrome (PMS), phobia including social phobia and agoraphobia, mania, manic-depression (bipolar disorder), smoking cessation and nicotine withdrawal syndrome. Each possibility represents a separate embodiment.

The term “trauma-related disorders” refers to nervous system injuries such as, for example, spinal cord injuries and various neurological disorders associated therewith. Examples of trauma-related disorders that may be treated with the compound or composition described herein include, but are not limited to, brain injury, ischemia-reperfusion injury, and stroke including ischemic stroke. Each possibility represents a separate embodiment.

The term “proliferative disorders” refers to a disorder in which a population of cells has become, in varying degrees, unresponsive to the control mechanisms that normally govern proliferation. In particular, the compounds and compositions disclosed herein are useful for the treatment of cancer. Cancer refers to various types of malignant neoplasms and tumors, including primary tumors, and tumor metastasis. Non-limiting examples of cancers which can be treated by the compounds and compositions of the present invention are brain, ovarian, colon, prostate, kidney, bladder, breast, lung, oral, and skin cancers. Each possibility represents a separate embodiment. Specific examples of cancers are carcinomas, sarcomas, myelomas, leukemias, lymphomas and mixed type tumors. Each possibility represents a separate embodiment. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Each possibility represents a separate embodiment. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, and hepatocarcinoma. Each possibility represents a separate embodiment.

In some representative embodiments, the cancer is selected from the group consisting of head and neck cancer, sarcoma, multiple myeloma, ovarian cancer, breast cancer, bladder cancer, kidney cancer, stomach cancer, hematopoietic cancers, lymphoma, leukemia, lung carcinoma, melanoma, glioblastoma, hepatocarcinoma, prostate cancer, pancreatic cancer, and colon cancer. Each possibility represents a separate embodiment.

In other representative embodiments, the cancer is selected from the group consisting of T cell acute lymphoblastic leukemia (T-ALL), chronic lymphocytic leukemia (CLL), melanoma, cholangiocarcinoma (CCC), colorectal cancer, lung adenocarcinoma, glioblastoma, renal cell carcinoma, ovarian cancer, prostate cancer, breast cancer, pancreatic ductal adenocarcinoma (PDAC), cervical cancer, head and neck squamous cell carcinomas (HNSCC), hepatocellular carcinoma (HCC), medulloblastoma, B cell acute lymphoblastic leukemia (B-ALL), acute myeloid leukemia (AML), small cell lung carcinoma (SCLC), non-small-cell lung carcinoma (NSCLC), lung squamous cell carcinoma (SqCC), cutaneous squamous cell carcinoma (SqCC), chronic myelomonocytic leukemia (CMML), cutaneous T-cell lymphoma (CTCL) including mycosis fungoides and Sézary syndrome, intestinal and diffuse-type gastric cancer, pilocytic astrocytoma (PA), choroid plexus tumor, laryngeal squamous cell carcinoma (LSCC), gallbladder carcinoma, Kaposi's sarcoma, B cell malignancy, T cell lymphoma, pancreatic cancer, nasopharyngeal carcinoma, squamous cell carcinoma, prostatic adenocarcinoma, infantile myofibromatosis (IM), lateral meningocele syndrome, and desmoid tumor. Each possibility represents a separate embodiment.

As used herein, the term “treating” includes, but is not limited to, preventing the disorder or disease from occurring in a subject or arresting the development or progression of the disorder or disease, relieving the disorder or disease, causing regression of the disorder or disease, relieving a condition caused by the disease or disorder, or ameliorating the symptoms of the disease or disorder. With reference to proliferative disorders, in particular cancer, the term “treating” includes, but is not limited to, at least one of the following: a decrease in the rate of growth of the cancer, cessation of cancerous growth, and, in preferred cases, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. It is to be understood that the term “treating cancer” also refers to the inhibition of a malignant (cancer) cell proliferation including tumor formation, primary tumors, tumor progression or tumor metastasis. The term “inhibition of proliferation” in relation to cancer cells, may further refer to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next.

In some embodiments, treatment comprises modulating (i.e. increasing or decreasing) the expression of a NOTCH protein, in particular the abnormal expression of a NOTCH protein. In some embodiments, the abnormal expression level relates to an increased NOTCH protein expression level. In some embodiments, the abnormal level of a NOTCH protein relates to a decreased expression level of a NOTCH protein. In some embodiments, the NOTCH protein is encoded by a NOTCH gene comprising at least one mutation. The mutation may be a missense mutation, a nonsense mutation, a frameshift mutation, a mutation that results in a shorter protein encoded from the mRNA harboring the mutation, or a mutation that renders a nonfunctional protein encoded from an mRNA harboring the mutation. Each possibility represents a separate embodiment.

As used herein, the term “administering” refers to bringing in contact with the compound and/or composition of the present invention. Administration can be accomplished to living organisms, for example humans.

A “therapeutic” treatment is a treatment administered to a subject who exhibits signs of pathology for the purpose of diminishing or eliminating those signs. A “therapeutically effective amount” is that amount of compound which is sufficient to provide a beneficial effect to the subject to which the compound is administered. The precise dose to be employed in the pharmaceutical composition comprising a compound of any of formulae I or 1-37 will depend on the route of administration, and the seriousness of the disease, and should be decided according to the judgment of the practitioner and each patient's circumstances. A preferred dosage will be within the range of about 0.01-1,000 mg/kg of body weight, about 0.1 mg/kg to 100 mg/kg, about 1 mg/kg to 100 mg/kg, about 10 mg/kg to 75 mg/kg, about 0.1 to 1 mg/kg etc., including each value within the specified ranges. Exemplary non-limiting amounts of the compound of any of formulae I or 1-37 include about 0.1 mg/kg, about 0.2 mg/kg, about 0.5 mg/kg, about 1 mg/kg, about 5 mg/kg, about 10 mg/kg, about 20 mg/kg, about 50 mg/kg, about 60 mg/kg, about 75 mg/kg, and about 100 mg/kg. Each possibility represents a separate embodiment. Alternatively, the amount administered can be measured and expressed as molarity of the administered compound. By way of illustration and not limitation, the compound of any of formulae I or 1-37 can be administered in a range of about 0.1 to 10 mM, including each value within the specified range e.g., about 0.1, 0.25, 0.5, 1 or 2 mM. Each possibility represents a separate embodiment. Alternatively, the amount administered can be measured and expressed as mg/ml, μg/ml, or ng/ml.

The administration schedule will depend on several factors such as the severity and progression of the disorder, age, weight etc. For example, the compositions of the invention can be taken once-daily, twice-daily, thrice daily, once-weekly or once-monthly. Each possibility represents a separate embodiment. In addition, the administration can be continuous, i.e., every day, or intermittent. The terms “intermittent” or “intermittently” as used herein means stopping and starting at either regular or irregular intervals. For example, intermittent administration can be administration one to six days per week or it may mean administration in cycles (e.g., daily administration for two to eight consecutive weeks, then a rest period with no administration for up to one week) or it may mean administration on alternate days. The effectiveness of said compositions could enable a shortened period of treatment with superior results.

Although the pharmaceutical composition comprising a compound of any of formulae I or 1-37 may be administered as the single therapeutic agent, combination therapy including co-administration with one or more additional agents is within the scope of the present invention. Co-administration of a compound of any of formulae I or 1-37 with one or more therapeutic agents may take place sequentially in any order, simultaneously or a combination thereof. For example, administration of a compound of any of formulae I or 1-37 can take place prior to, after or at the same time as the administration of the additional therapeutic agent(s). For example, a total treatment period can be decided for the compound of any of formulae I or 1-37. The additional agent(s) can be administered prior to the onset of treatment with the compound of any of formulae I or 1-37 or following treatment with the compound of any of formulae I or 1-37. In addition, the additional agent(s) can be administered during the period of administering the compound of any of formulae I or 1-37 but does not need to occur over the entire treatment period. In another embodiment, the treatment regimen includes pre-treatment with one agent, followed by the addition of the other agent or agents. Alternating sequences of administration are also contemplated. Alternating administration includes administration of a compound of any of formulae I or 1-37, followed by the additional agent, followed by a compound of any of formulae I or 1-37, etc. The aforementioned sequences can also be administrated in several cycles wherein each cycle may be similar or different with each possibility representing a separate embodiment. In one embodiment, the therapeutic efficacy of the combination of a compound of any of formulae I or 1-37 and the additional agent(s) is additive. In other embodiments, the therapeutic efficacy is synergistic, namely the overall dose of each of the components may be lower, thus resulting in significantly lower side effects experienced by the subject, while a sufficient desirable therapeutic effect is nonetheless achieved. When combination therapy is involved, the compound of any of formulae I or 1-37 and the additional therapeutic agent(s) may be provided in a single dosage form such as a fixed-dose combination or in separate compositions intended for simultaneous administration.

The one or more additional therapeutic agents include, but are not limited to, cannabinoids, terpenes, terpenoids, flavonoids, oils, nitrogenous compounds, amino acids, proteins, glycoproteins, sugars, hydrocarbons, fatty acids, esters, lactones, steroids, non-cannabinoid phenols, and a mixture or combination thereof. Each possibility represents a separate embodiment.

In some aspects and embodiments, the compounds and compositions of the present invention are administered in combination with at least one cannabinoid. Exemplary cannabinoids that can be used in the combination therapy include, but are not limited to, cannabidivarinic acid (CBDVA), cannabidiolic acid (CBDA), cannabigerolic acid (CBGA), cannabidiol (CBD), cannabinol (CBN), cannabinolic acid (CBNA), tetrahydrocannabinol (THC), cannabichromene (CBC), cannabichromenic acid (CBCA), tetrahydrocannabinolic acid (THCA), cannabicitran, tetrahydrocannabivarin (THCV), cannabigerol (CBG), sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabigerolic acid monomethyl ether (CBGMA), cannabigerol monomethyl ether (CBGM), cannabicyclol (CBL), cannabicyclolic acid (CBLA), THCA-C4, THC-C4, tetrahydrocannabivarin carboxylic acid (THCVA), tetrahydrocannabivarin (THCV), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol (CBN), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, epicannabidiol, and a mixture or combination thereof. Each possibility represents a separate embodiment. In one embodiment, the compounds and compositions of the present invention are administered in combination with cannabidiol (CBD).

When the compound of the present invention is co-administered with at least one cannabinoid, typical ratios of the compound of the present invention and the at least one cannabinoid include, but are not limited to, about 1:1,000 to about 1,000:1, including all iterations of ratios within the specified range. Exemplary ratios include, but are not limited to, about 1:1,000, about 1:900, about 1:800, about 1:700, about 1:600, about 1:500, about 1:400, about 1:300, about 1:200, about 1:100, about 1:75: about 1:50, about 1:40, about 1:30, about 1:20, about 1:15, about 1:10, about 1:5, about 1:2, about 1:1, about 2:1, about 5:1, about 10:1, about 15:1, about 20:1, about 30:1, about 40:1, about 50:1, about 75:1, about 100:1, about 200:1, about 300:1, about 400:1, about 500:1, about 600:1, about 700:1, about 800:1, about 900:1, or about 1,000:1. Each possibility represents a separate embodiment.

In some aspects and embodiments, the compounds and compositions of the present invention are administered in combination with at least one anti-cancer agent. Exemplary anti-cancer agents include, but are not limited to, a modulator of a protein kinase (PK), an inhibitor of mammalian target of rapamycin (mTOR), a mitogen-activated protein kinase (MEK) inhibitor, a mutated B-Raf inhibitor, a chemotherapeutic agent, an antibody against PDLs, PD1, and/or CTLA4, and combinations thereof. Each possibility represents a separate embodiment.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the use of “a” and “an” means “at least one” or “one or more” unless the context clearly dictates otherwise. It should be noted that the term “and” or the term “or” are generally employed in their sense including “and/or” unless the context clearly dictates otherwise.

As used herein, when a numerical value is preceded by the term “about”, the term “about” is intended to indicate ±10%.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### Example 1: Synthesis

### Preparation of a Compound of Formula 1:

A compound of formula 1 was prepared according to the following scheme:

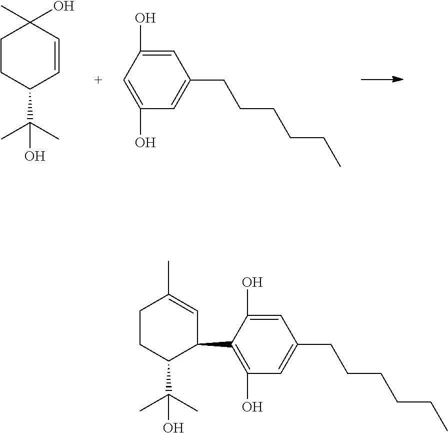 [SMILES: CC1(O)C=C[C@H](C(C)(C)O)CC1 ; CCCCCCc1cc(O)cc(O)c1 ; CCCCCCc1cc(O)c([C@@H]2C=C(C)CC[C@H]2C(C)(C)O)c(O)c1]

In particular, 1,3-benzelenediol,5-hexyl (780 mg, 4 mmol), cis-p-menth-2-ene-1,8-diol (680 mg, 4 mmol) and p-TSA (25 mg, 4% eq) in DCM (27 ml) were stirred for 24 hr at RT under inert atmosphere. The reaction mixture was diluted with DCM (20 ml) and washed with 0.5N NaOH (20 ml), 1N HCl (20 ml), and brine. An organic solution was separated, dried over sodium sulfate and the solvent was evaporated to dryness to give 970 mg of the crude product. Purification of the crude product was performed by flash column chromatography to give 526 mg of the compound.

### Preparation of a Compound of Formula 2:

A compound of formula 2 was prepared according to the following scheme:

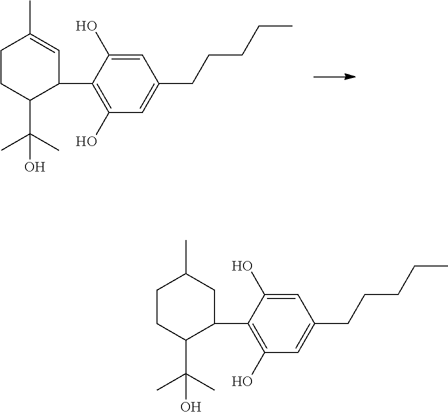 [SMILES: CCCCCc1cc(O)c(C2C=C(C)CCC2C(C)(C)O)c(O)c1 ; CCCCCc1cc(O)c(C2CC(C)CCC2C(C)(C)O)c(O)c1]

In particular, a solution of 2-[6-(2-hydroxypropan-2-yl)-3-methylcyclohex-2-en-1-yl]-5-pentylbenzene-1,3-diol (166 mg, 0.5 mmol), ammonium formate (320 mg) and 10% Pd/C (20 mg) in methanol (3 ml) was stirred for 2 days at RT. The reaction mixture was added to water (50 ml) followed by the addition of 1N HCl to a pH of 1-2. The crude product was extracted with TBME (30 ml). The crude product (180 mg) was purified by flash column chromatography to give 63 mg of a mixture of the a and 3-Me isomers (33/66%).

Alternatively, 2-[6-(2-hydroxypropan-2-yl)-3-methylcyclohex-2-en-1-yl]-5-pentylbenzene-1,3-diol (0.5 g, 1.5 mmol) was dissolved in ethanol (5 ml) and 10% Pd/C (50 mg) was introduced under inert atmosphere. Hydrogen (gas) was babbled into the reaction mixture for 2-3 hr at RT. The reaction mixture was filtered and ethanol was evaporated to dryness to give 0.5 g of a semi solid product.

### Preparation of a Compound of Formula 3:

A compound of formula 3 was prepared from CBDV according to the following scheme:

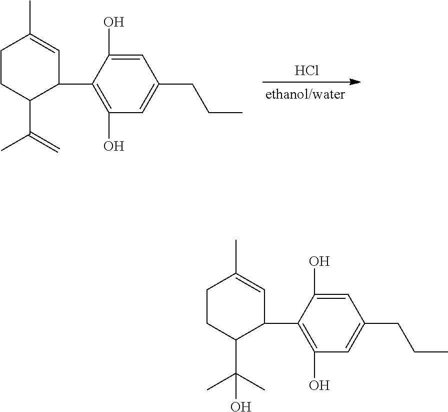 [SMILES: C=C(C)C1CCC(C)=CC1c1c(O)cc(CCC)cc1O ; Cl ; CCCc1cc(O)c(C2C=C(C)CCC2C(C)(C)O)c(O)c1]

In particular, CBDV (1.0 g, 3.5 mmol) was stirred in a mixture of ethanol:water: 37% HCl (65:25:10 v/v, 10 ml) for 3 hr at RT. A product was extracted with hexane (20 ml×2). The organic phase was separated, washed with water (20 ml) and dried over sodium sulfate. The product was separated by flash column chromatography to give 50 mg of a white solid.

¹H NMR (400 MHz, CDCl₃) δ 6.48 (1H), 6.26-6.30 (2H, Ar), 5.67 (1H, C═CH), 3.84 (1H), 2.43 (2H, CH₂), 2.08-2.11 (2H), 1.88-1.91 (3H), 1.80 (3H, Me), 1.68-17.3 (1H), 1.56-1.62 (2H), 1.27 (1H), 1.24 (6H, 2 Me), 0.91 (3H, Me).

### Preparation of a Compound of Formula 4:

A compound of formula 4 was prepared according to the following scheme:

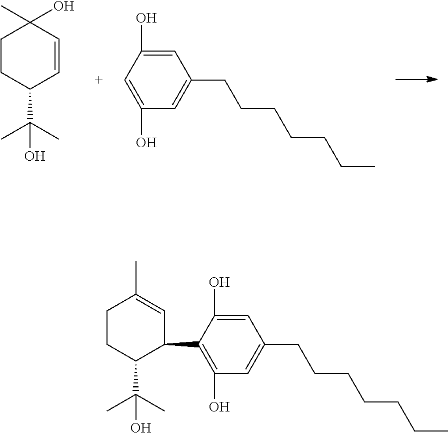 [SMILES: CC1(O)C=C[C@H](C(C)(C)O)CC1 ; CCCCCCCc1cc(O)cc(O)c1 ; CCCCCCCc1cc(O)c([C@@H]2C=C(C)CC[C@H]2C(C)(C)O)c(O)c1]

In particular, 1,3-benzelenediol,5-heptyl (832 mg, 4 mmol), cis-p-menth-2-ene-1,8-diol (680 mg, 4 mmol) and p-TSA (25 mg, 4% eq) in DCM (27 ml) were stirred for 24 hr at RT under inert atmosphere. The reaction mixture was diluted with DCM (20 ml) and washed with 0.5N NaOH (20 ml), 1N HCl (20 ml), and brine. An organic solution was separated, dried over sodium sulfate and the solvent was evaporated to dryness to give 990 mg of the crude product. Purification of the crude product was performed by flash column chromatography to give 550 mg of the compound.

### Preparation of a Compound of Formula 7:

A compound of formula 7 was prepared according to the following scheme:

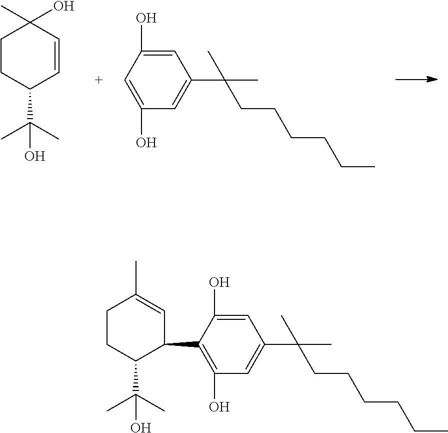 [SMILES: CC1(O)C=C[C@H](C(C)(C)O)CC1 ; CCCCCCC(C)(C)c1cc(O)cc(O)c1 ; CCCCCCC(C)(C)c1cc(O)c([C@@H]2C=C(C)CC[C@H]2C(C)(C)O)c(O)c1]

In particular, 5-(1,1-dimethylheptyl)-resorcinol (710 mg, 3 mmol), cis-p-menth-2-ene-1,8-diol (510 mg, 3 mmol) and p-TSA (25 mg, 4% eq) in DCM (25 ml) were stirred for 24 hr at RT under inert atmosphere. The reaction mixture was diluted with DCM (20 ml) and washed with 0.5N NaOH (20 ml), 1N HCl (20 ml) and brine. An organic solution was separated, dried over sodium sulfate and the solvent was evaporated to dryness to give 1,300 mg of the crude product. Purification of the crude product was performed by flash column chromatography to give 600 mg of the compound.

### Preparation of a Compound of Formula 11:

A compound of formula 11 was prepared according to the following scheme:

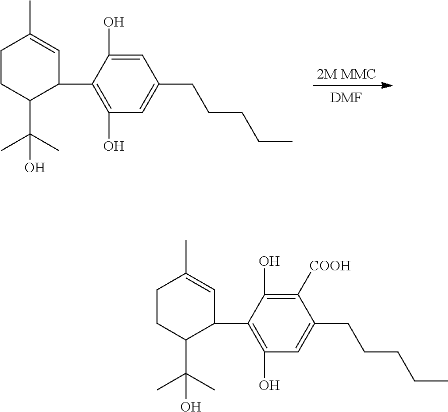

In particular, a solution of 2-[6-(2-hydroxypropan-2-yl)-3-methylcyclohex-2-en-1-yl]-5-pentylbenzene-1,3-diol (0.7 g, 2.1 mmol) in 2.0 M solution magnesium methyl carbonate in DMF (2M MMC, 10 ml) was heated for 1 hr at 125° C. under inert atmosphere. The reaction mixture was cooled to 50-60° C. and diluted with toluene (20 ml). The solution was slowly added to cold 1N HCl (50 ml) with effective stirring and the two phases mixture was stirred for 15-20 min. The organic phase was separated and washed with water (20 ml). The product was extracted with 1N NaOH (25 ml×2). The water phase was separated and 37% HCl was added dropwise to pH=1-2. The product was extracted with TBME (25 ml×2). The organic phase was washed with water and dried over sodium sulfate. The product was purified by flash column chromatography to give 240 mg of an off-white solid.

¹H NMR (400 MHz, CDCl₃) δ 6.23 (1H, Ar), 5.56 (1H, C═CH), 4.06 (1H), 2.7, 2.8 (2H), 2.11 (2H), 1.91 (2H), 1.8 (3H, Me), 1.46-1.66 (4H), 1.25 (6H), 0.86 (3H, Me).

### Preparation of a Compound of Formula 12:

A compound of formula 12 was prepared from a compound of formula 11 according to the following scheme:

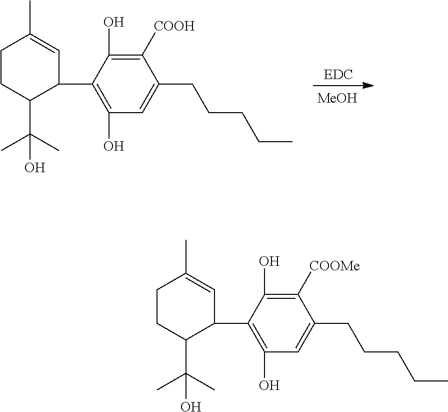 [SMILES: "MeOH" ; CCCCCc1cc(O)c(C2C=C(C)CCC2C(C)(C)O)c(O)c1C(=O)O ; "EDC" ; CCCCCc1cc(O)c(C2C=C(C)CCC2C(C)(C)O)c(O)c1C(=O)OC]

In particular, a compound of formula 11 (300 mg, 0.8 mmol) was dissolved in methanol and 1-ethyl-3-(3-dimethylaminopropyl) carbodiimide (EDC) (150 mg) was added. The reaction mixture was stirred for 1 hr at RT under inert atmosphere and methanol was evaporated to dryness. The residue oil was dissolved in a mixture of water (20 ml) and MTBE (30 ml). The organic phase was separated, dried over sodium sulfate and evaporated to dryness. The crude product was purified by flash column chromatography to give 330 mg of a white solid.

¹H NMR (400 MHz, CDCl₃) δ 6.25 (1H, Ar), 5.44 (1H, C═CH), 4.1 (1H), 3.91 (3H, CH₃—O—), 2.71-2.81 (2H), 2.11 (2H), 1.98 (1H), 1.91 (1H), 1.77 (3H, Me), 1.56 (1H), 1.50 (2H), 1.33 (4H), 1.16-1.21 (6H, 2Me), 0.9 (3H, Me).

### Preparation of a Compound of Formula 13:

A compound of formula 13 was prepared from a compound of formula 11 according to the following scheme:

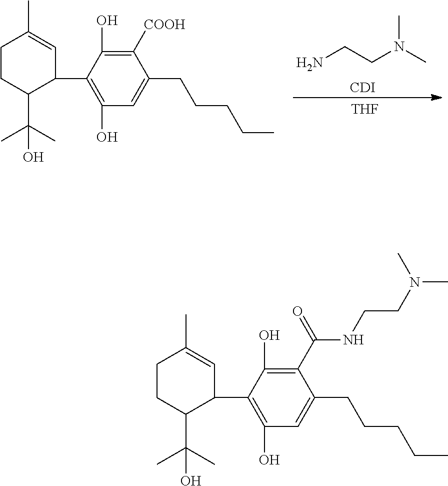

In particular, a compound of formula 11 (100 mg, 0.3 mmol) was dissolved in THF (1.5 ml) and carbonyldiimidazole (CDI) (105 mg, 0.64 mmol) was added. The reaction mixture was stirred for 1 hr at 47° C. and N,N-dimethylethylene-diamine (80 mg, 0.9 mmol) was introduced. The solution was stirred for 18 hr at 47° C. The reaction mixture was washed with brine (5 ml) and the product was extracted with MTBE (5 ml). The organic phase was separated, dried over sodium sulfate and the solvent was evaporated to dryness to give 90 mg of the crude product. The crude product was purified by flash column chromatography to give 39 mg of a white semi-solid product.

### Preparation of a Compound of Formula 14:

A compound of formula 14 was prepared from a compound of formula 11 according to the following scheme:

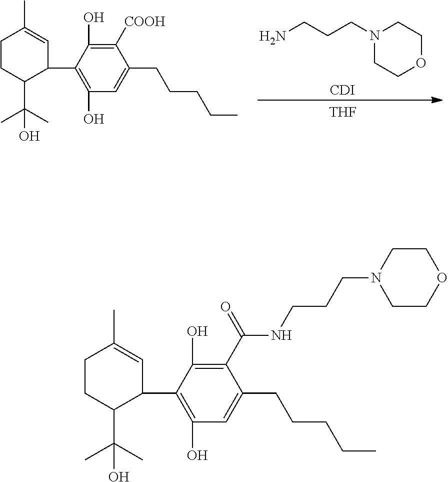 [SMILES: CCCCCc1cc(O)c(C2C=C(C)CCC2C(C)(C)O)c(O)c1C(=O)O ; NCCCN1CCOCC1 ; "CDI" ; "THF" ; CCCCCc1cc(O)c(C2C=C(C)CCC2C(C)(C)O)c(O)c1C(=O)NCCCN1CCOCC1]

In particular, a compound of formula 11 (120 mg, 0.32 mmol) was dissolved in THF (1.5 ml) and carbonyldiimidazole (CDI) (105 mg, 0.64 mmol) was added. The reaction mixture was stirred for 1 hr at 47° C. and 3-morpholinpropyl-amine (140 mg, 0.96 mmol) was introduced. The solution was stirred for 2 hr at 47° C. The reaction mixture was washed with brine (5 ml) and the product was extracted with MTBE (5 ml). The organic phase was separated, dried over sodium sulfate and the solvent was evaporated to dryness to give 100 mg of the crude product. The crude product was purified by flash column chromatography to give 39 mg of a white semi-solid product.

### Preparation of a Compound of Formula 17:

A compound of formula 17 was prepared in several steps. First, a solution of 2-[6-(2-hydroxypropan-2-yl)-3-methylcyclohex-2-en-1-yl]-5-pentylbenzene-1,3-diol (1.4 g, 4.2 mmol) in pyridine (10 ml) was prepared and acetic anhydride (2 ml) was added. The solution was stirred for 3 days at RT under inert atmosphere. The reaction mixture was then diluted with 1 N HCl (100 ml), and the product was extracted with TBME (50 ml×2). The organic layer was separated, washed with water, and dried over sodium sulfate. The suspension was filtered and the solvent was evaporated to dryness to give 2.0 g (100%) of the triacetylated compound:

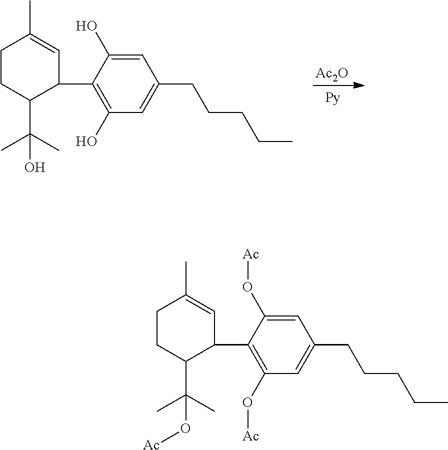 [SMILES: "Ac2O" ; CCCCCc1cc(O)c(C2C=C(C)CCC2C(C)(C)O)c(O)c1 ; "Py" ; CCCCCc1cc(OC(C)=O)c(C2C=C(C)CCC2C(C)(C)OC(C)=O)c(OC(C)=O)c1]

The triacetylated compound (2.0 g) was then dissolved in a mixture of acetic acid (8.5 ml) and acetic anhydride (4.25 ml) followed by the addition of potassium dichromate (2.2 g). The reaction mixture was stirred for 24 hr at 38° C. (bath) and diluted with water (200 ml). The product was extracted with TBME (50 ml×3). The organic layer was separated, washed with brine (50 ml), and dried over sodium sulfate. The suspension was filtered and the solvent was evaporated to dryness to give 1.2 g (60%) of the triacetylated keto product. The product was separated to three fractions A (200 mg), B (350 mg), and C (67 mg) by flash column chromatography. Fraction B was used in the subsequent step.

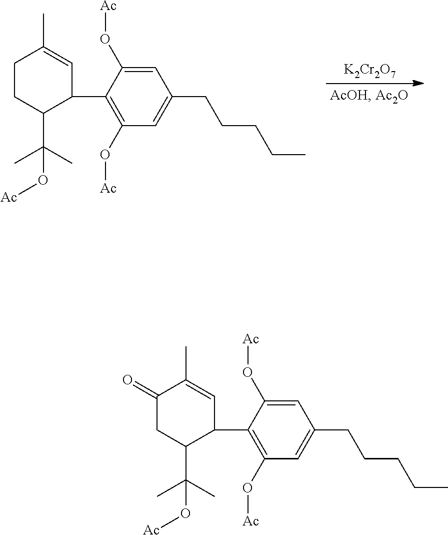

Finally, the triacetylated keto product (fraction B, 350 mg) was dissolved in MeOH (9 ml) and sodium borohydride (0.3 g) was added in portions at 0-5° C. under inert atmosphere. The reaction mixture was stirred for 24 hr and added to brine (50 ml). 1 N HCl was added to a pH of 1-2 and the product (compound 17) was extracted with TBME (10 ml×3). The organic layer was separated, washed with brine (20 ml), and dried over sodium sulfate. The suspension was filtered and the solvent was evaporated to dryness to give 0.32 g of compound 17.

¹H NMR (500 MHz, CDCl₃) δ 6.26 (2H, Ar), 5.50 (1H, C═CH), 4.33 (1H, CH), 3.99 (1H, CH), 2.43 (2H), 2.30 (1H, CH₂), 1.86 (3H, CH₃), 1.64 (1H, CH₂), 2.11 (1H, CH), 1.55 (2H, CH₂), 1.29-1.30 (4H), 1.26 (3H, CH₃), 1.18 (3H, CH₃), 0.87 (3H, Me).

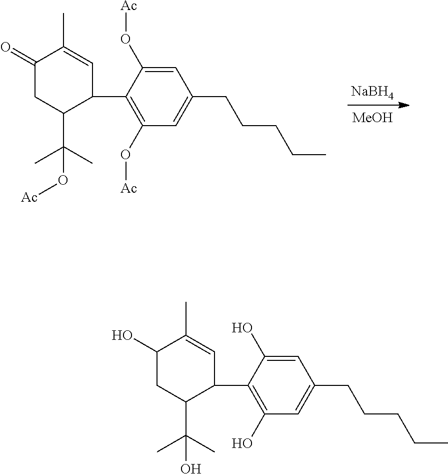 [SMILES: "MeOH" ; CCCCCc1cc(OC(C)=O)c(C2C=C(C)C(=O)CC2C(C)(C)OC(C)=O)c(OC(C)=O)c1 ; "NaBH4" ; CCCCCc1cc(O)c(C2C=C(C)C(O)CC2C(C)(C)O)c(O)c1]

### Preparation of a Compound of Formula 30:

A compound of formula 30 was prepared according to the following scheme:

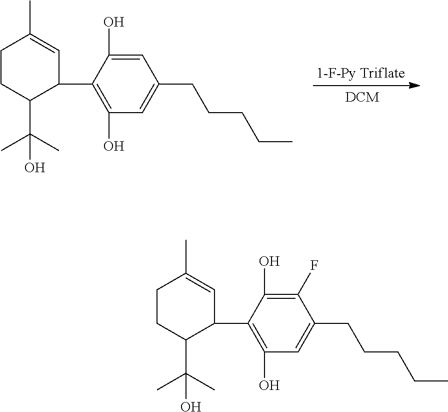

In particular, a solution of 2-[6-(2-hydroxypropan-2-yl)-3-methylcyclohex-2-en-1-yl]-5-pentylbenzene-1,3-diol (0.332 g, 1 mmol) and 1-fluor-pyridinium triflate (0.272 g, 1.1 mmol) in DCM (13 ml) was stirred for 18 hr at RT under inert atmosphere. The reaction mixture was diluted with DCM (10 ml) and the organic phase was washed with a solution of 5% sodium bicarbonate in water (20 ml). The organic phase was separated, and the solvent was evaporated to dryness. A crude product was purified by flash column chromatography to give 207 mg of a mixture of the starting material with mono-fluor and di-fluor derivatives. This mixture was separated by HPLC preparative.

¹H NMR (400 MHz, CDCl₃) δ 6.22, (1H, Ar), 5.56 (1H, C═CH), 3.88 (1H), 2.52 (2H), 2.14, 2.09 (2H), 1.98, 1.66 (2H), 1.80 (3H, Me), 1.93 (1H), 1.57 (2H), 1.32 (4H), 1.22 (6H, 2Me), 0.88 (3H, Me).

### Preparation of a Compound of Formula 37:

A compound of formula 37 was prepared according to the following scheme:

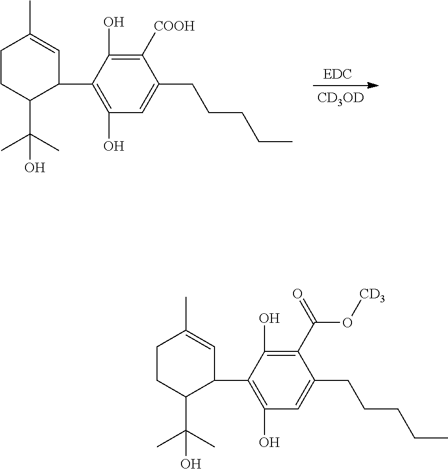 [SMILES: CCCCCc1cc(O)c(C2C=C(C)CCC2C(C)(C)O)c(O)c1C(=O)O ; "CD3OD" ; "EDC" ; [2H]C([2H])([2H])OC(=O)c1c(CCCCC)cc(O)c(C2C=C(C)CCC2C(C)(C)O)c1O]

In particular, 2-[6-(2-hydroxypropan-2-yl)-3-methylcyclohex-2-en-1-yl]-5-pentylbenzene-1,3-diol (94 mg, 0.25 mmol) was dissolved in CD₃OD and 1-ethyl-3-(3-dimethylaminopropyl) carbodiimide (EDC) (60 mg) was added. The reaction mixture was stirred overnight at RT under inert atmosphere and CD₃OD was evaporated to dryness. A residue oil was dissolved in a mixture of water (10 ml) and MTBE (3 ml). The organic phase was separated, dried over sodium sulfate and evaporated to dryness. A crude product was purified by flash column chromatography to give 70 mg of a white solid.

### Example 2: Steroidogenesis

The effect of the compounds according to certain embodiments of the present invention as inhibitors of steroidogenesis was assessed. Human adrenal cells were thawed and cultured in a suitable medium at a final concentration of 5×10⁵ cells per mL (performed in triplicates). Following 24 hours of incubation, cells were treated with 10 μM Forskolin. After 48 hours of incubation, cells were treated with controls (1 μM Prochloraz, and 0.2 μg/mL or 1 μg/mL CBD) and compounds according to embodiments of the present invention for 24 hours. Next, supernatants were collected, and the secretion levels of cortisol were measured by ELISA (R&D systems). Optical density was determined by ‘CLARIOStar-plus’® plate-reader. Cortisol concentration (ng/ml) was calculated using a standard curve. The results are summarized in Table 1:

TABLE 1

| | | | | Cortisol | |
|---|---|---|---|---|---|
| Treatment | Inhibitor | | | ng/mL | % of Forskolin |
| No treatment | | | | 10 | 9% |
| Forskolin 10 μM | | | | 107 | 100% |
| Forskolin 10 μM | Prochloraz | 1 | μg/mL | 23 | 22% |
| | CBD | 0.2 | μg/mL | 101 | 95% |
| | | 1 | μg/mL | 62 | 58% |
| | Formula 14 | 0.2 | μg/mL | 41 | 39% |
| | | 1 | μg/mL | 45 | 42% |
| | Formula 12 | 0.2 | μg/mL | 43 | 41% |
| | | 1 | μg/mL | 45 | 42% |
| | Formula 7 | 0.2 | μg/mL | 39 | 37% |
| | | 1 | μg/mL | 47 | 44% |
| | Formula 4 | 0.2 | μg/mL | 38 | 36% |
| | | 1 | μg/mL | 35 | 32% |
| | Formula 1 | 0.2 | μg/mL | 37 | 35% |
| | | 1 | μg/mL | 24 | 22% |
| | Formula 2 | 0.2 | μg/mL | 100 | 94% |
| | | 1 | μg/mL | 77 | 72% |

Thus, the compounds of the present invention are shown to inhibit steroidogenesis thereby reducing cortisol secretion.

### Example 3: Cancer

The effect of the compounds according to certain embodiments of the present invention as anti-cancer agents was assessed. Human cancer cells of prostate (PC3 cell line), bladder (T24 cell line), or breast (MDA-MB-231 cell line) cancers were thawed and cultured in suitable media at a final concentration of 3×10⁵ cells/mL and tested in triplicates. Following 24 hours of incubation, cells were treated with the tested compounds at 2 μg/ml and 4 μg/ml concentrations or controls (cells were grown in starvation medium and treated with Mitomycin C). Mitomycin C is a potent antineoplastic agent that creates DNA cross-linkage resulting in cell arrest and consequently cell death. It is used to treat various types of cancers such as bladder and breast cancers. In in-vitro models, it acts as a proliferation inhibitor as it induces G0-G1 cell growth arrest. The number of cells in each sample was quantified immediately upon treatment (time-point 0) and every 24 hours until full cell confluency was reached. 24 hours post-treatment, PI (1 μg/ml) was added to the treated cells for 1 hour. Using cell-by-cell analysis (IncuCyte™ cell imager), the PI-positive cells were counted and compared to the relative number of cells in the non-treated (NT) wells. The results are summarized in Tables 2-4:

TABLE 2
Prostate cancer

| | Number of | | |
|---|---|---|---|
| | cells | SE | % NT |
| NT | 25.07 | 2.95 | 100% |
| CBD (2 μg/ml) | 22.53 | 3.01 | 90% |
| CBD (4 μg/ml) | 80.13 | 7.82 | 320% |
| Formula 12 (2 μg/ml) | 53.93 | 6.20 | 215% |
| Formula 12 (4 μg/ml) | 97.80 | 6.96 | 390% |
| Formula 2 (2 μg/ml) | 76.93 | 10.67 | 307% |
| Formula 2 (4 μg/ml) | 90.93 | 8.78 | 363% |
| Formula 7 (2 μg/ml) | 81.80 | 7.93 | 326% |
| Formula 7 (4 μg/ml) | 1037.53 | 96.35 | 4139% |
| Formula 4 (2 μg/ml) | 57.27 | 6.81 | 228% |
| Formula 4 (4 μg/ml) | 704.33 | 47.72 | 2810% |
| Formula 1 (2 μg/ml) | 54.13 | 5.56 | 216% |
| Formula 1 (4 μg/ml) | 65.27 | 8.34 | 260% |

TABLE 3
Bladder cancer

| | Number of | | |
|---|---|---|---|
| | cells | SE | % NT |
| NT | 109.80 | 9.34 | 100% |
| CBD (2 μg/ml) | 171.47 | 11.16 | 156% |
| CBD (4 μg/ml) | 1969.93 | 108.68 | 1794% |
| Formula 7 (2 μg/ml) | 470.67 | 34.05 | 429% |
| Formula 7 (4 μg/ml) | 1269.33 | 118.91 | 1156% |
| Formula 4 (2 μg/ml) | 118.20 | 6.50 | 108% |
| Formula 4 (4 μg/ml) | 771.60 | 98.84 | 703% |

TABLE 4
Breast cancer

| | Number of | | |
|---|---|---|---|
| | cells | SE | % NT |
| NT (100 mg/ml) | 51.00 | 2.14 | 100% |
| CBD (2 μg/ml) | 89.40 | 7.78 | 175% |
| CBD (4 μg/ml) | 675.73 | 50.50 | 1325% |
| Formula 7 (2 μg/ml) | 141.47 | 8.84 | 277% |
| Formula 7 (4 μg/ml) | 1120.53 | 44.61 | 2197% |
| Formula 4 (2 μg/ml) | 61.07 | 6.66 | 120% |
| Formula 4 (4 μg/ml) | 722.47 | 79.11 | 1417% |

Thus, the compounds of the present invention are potent anti-cancer agents against various cancer types.

While certain embodiments of the invention have been illustrated and described, it is to be clear that the invention is not limited to the embodiments described herein. Numerous modifications, changes, variations, substitutions and equivalents will be apparent to those skilled in the art without departing from the spirit and scope of the present invention as described by the claims, which follow.

1-48. (canceled)

49. A compound represented by the structure of formula I: 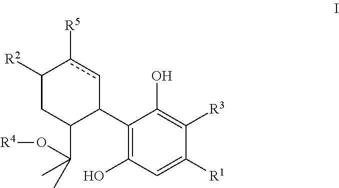
wherein
R¹ is C₁-C₁₂ alkyl, C₂-C₁₂ alkenyl, or C₂-C₁₂ alkynyl;
R² is H, deuterium, hydroxyl, halogen, haloalkyl, nitro, cyano, heterocyclyl, O-heterocyclyl, amino, or amido;
R³ is H, deuterium, hydroxyl, halogen, haloalkyl, nitro, alkoxy, aryloxy, amino, amido, or C(O)OR^{a} wherein R^{a} is H, deuterium, or C₁-C₄ alkyl;
R⁴ is H, deuterium, C₁-C₆ alkyl, C₂-C₆ alkenyl, C₂-C₆ alkynyl, aryl, or unsubstituted C(O)C₁-C₄ alkyl;
R⁵ is C₁-C₄ alkyl, C₂-C₄ alkenyl, C₂-C₄ alkynyl, or C(O)OR^{b} wherein R^{b} is H, deuterium, or C₁-C₄ alkyl; and
the dotted line represents an optional second carbon-carbon bond, wherein each of the alkyl, alkenyl, alkynyl, cyclyl, aryl, acyl, amino or amido is optionally deuterated, with the proviso that when R², R³, and R⁴ are H, R⁵ is methyl, and the dotted line represents a second carbon-carbon bond, then R¹ is not n-pentyl or 2-phenylethyl, including salts, hydrates, solvates, polymorphs, optical isomers, geometrical isomers, enantiomers, diastereomers, and mixtures thereof.

50. The compound of claim 49, wherein R¹ is C₁-C₁₂ alkyl; or wherein R¹ is selected from the group consisting of: propyl, butyl, pentyl, hexyl, heptyl, octyl, 1,1-dimethyl pentyl, 1-methyl pentyl, 1-methyl heptyl, 1,1-dimethyl heptyl, and 1-phenyl ethyl.

51. The compound of claim 49, wherein R² is hydrogen; or wherein R² is halogen; or wherein R² is O-heterocyclyl, wherein the O-heterocyclyl is O-glucosyl.

52. The compound of claim 49, wherein R³ is hydrogen; or wherein R³ is halogen; or wherein R³ is fluorine; or wherein R³ is C(O)OH; or wherein R³ is C(O)OCH₃ or C(O)OCD₃; or wherein R³ is a —C(O)NHR^{c} amido, wherein R^{c} is an alkyl-amino or an alkyl-heterocyclyl.

53. The compound of claim 49, wherein R⁴ is hydrogen; or wherein R⁴ is C₁-C₆ alkyl.

54. The compound of claim 49, wherein R⁵ is C₁-C₄ alkyl; or wherein R⁵ is methyl; or wherein R⁵ is hydroxymethyl.

55. The compound of claim 49, wherein R¹ is pentyl, R² and R⁴ are each H, R³ is halogen, R⁵ is methyl, and the dotted line represents a second carbon-carbon bond; or wherein R¹ is pentyl, R² and R⁴ are each H, R³ is fluorine, R⁵ is methyl, and the dotted line represents a second carbon-carbon bond; or wherein R¹ is propyl, R², R³, and R⁴ are each H, R⁵ is methyl, and the dotted line represents a second carbon-carbon bond; or wherein R¹ is hexyl, R², R³, and R⁴ are each H, R⁵ is methyl, and the dotted line represents a second carbon-carbon bond; or wherein R¹ is heptyl, R², R³, and R⁴ are each H, R⁵ is methyl, and the dotted line represents a second carbon-carbon bond; or wherein R¹ is 1,1-dimethyl heptyl, R², R³, and R⁴ are each H, R⁵ is methyl, and the dotted line represents a second carbon-carbon bond; or wherein R¹ is pentyl, R² is fluorine, R³ and R⁴ are each H, R⁵ is methyl, and the dotted line represents a second carbon-carbon bond; or wherein R¹ is pentyl, R² and R⁴ are each H, R³ is C(O)OH, R⁵ is methyl, and the dotted line represents a second carbon-carbon bond; or wherein R¹ is pentyl, R² and R⁴ are each H, R³ is C(O)OCH₃, R⁵ is methyl, and the dotted line represents a second carbon-carbon bond; or wherein R¹ is pentyl, R² and R⁴ are each H, R³ is C(O)OCD₃, R⁵ is methyl, and the dotted line represents a second carbon-carbon bond; or wherein R¹ is pentyl, R², R³, and R⁴ are each H, R⁵ is methyl, and the dotted line is absent; or wherein R¹ is pentyl, R² and R⁴ are each H, R³ is a —C(O)NHR^{c} amido, wherein R is -ethyldimethylamino, R⁵ is methyl, and the dotted line represents a second carbon-carbon bond; or wherein R¹ is pentyl, R² and R⁴ are each H, R³ is a —C(O)NHR^{c} amido, wherein R^{c} is -propylmorpholino, R⁵ is methyl, and the dotted line represents a second carbon-carbon bond.

56. The compound of claim 49, selected from the group consisting of: 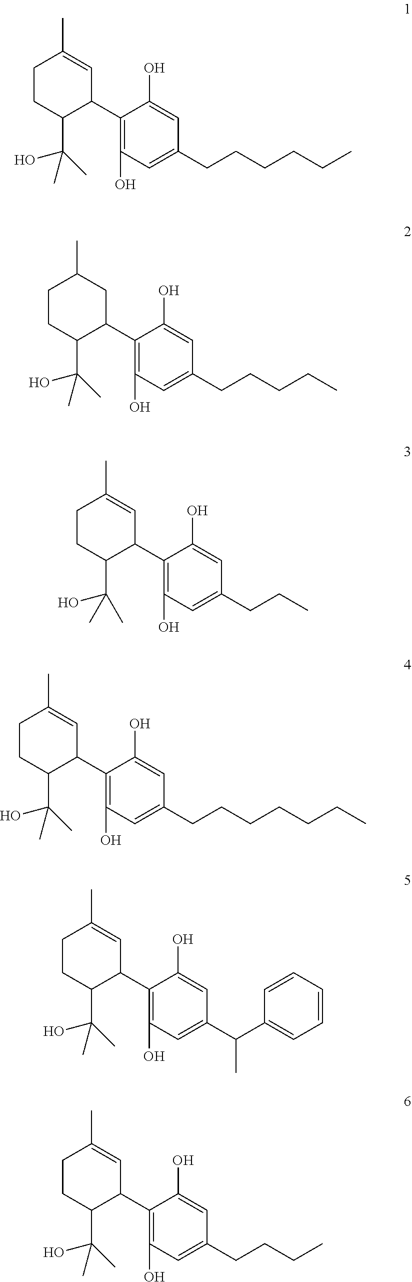 [SMILES: CCCCCCc1cc(O)c(C2C=C(C)CCC2C(C)(C)O)c(O)c1 ; CCCCCc1cc(O)c(C2CC(C)CCC2C(C)(C)O)c(O)c1 ; CCCc1cc(O)c(C2C=C(C)CCC2C(C)(C)O)c(O)c1 ; CCCCCCCc1cc(O)c(C2C=C(C)CCC2C(C)(C)O)c(O)c1 ; CC1=CC(c2c(O)cc(C(C)c3ccccc3)cc2O)C(C(C)(C)O)CC1 ; CCCCc1cc(O)c(C2C=C(C)CCC2C(C)(C)O)c(O)c1] 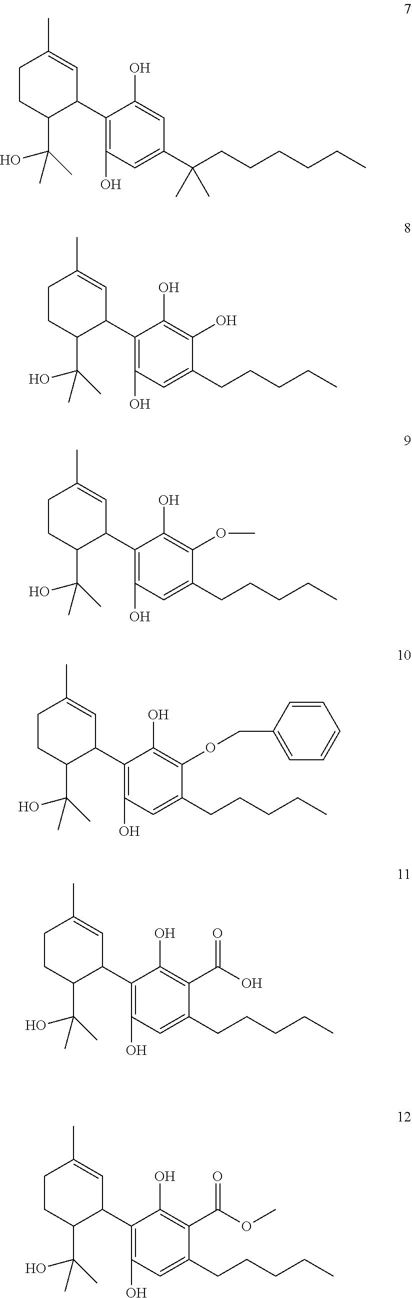 [SMILES: CCCCCCC(C)(C)c1cc(O)c(C2C=C(C)CCC2C(C)(C)O)c(O)c1 ; CCCCCc1cc(O)c(C2C=C(C)CCC2C(C)(C)O)c(O)c1O ; CCCCCc1cc(O)c(C2C=C(C)CCC2C(C)(C)O)c(O)c1OC ; CCCCCc1cc(O)c(C2C=C(C)CCC2C(C)(C)O)c(O)c1OCc1ccccc1 ; CCCCCc1cc(O)c(C2C=C(C)CCC2C(C)(C)O)c(O)c1C(=O)O ; CCCCCc1cc(O)c(C2C=C(C)CCC2C(C)(C)O)c(O)c1C(=O)OC] 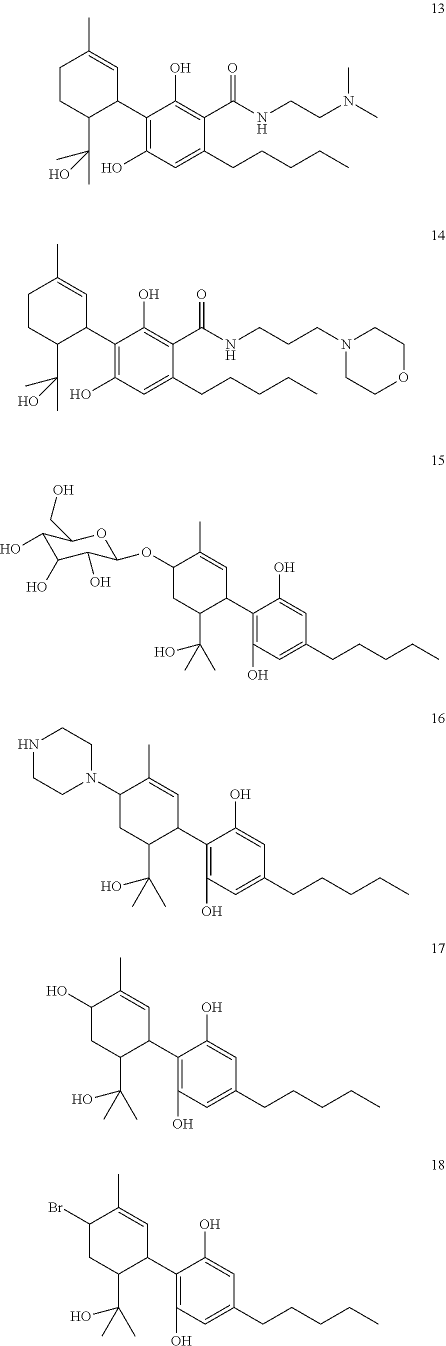 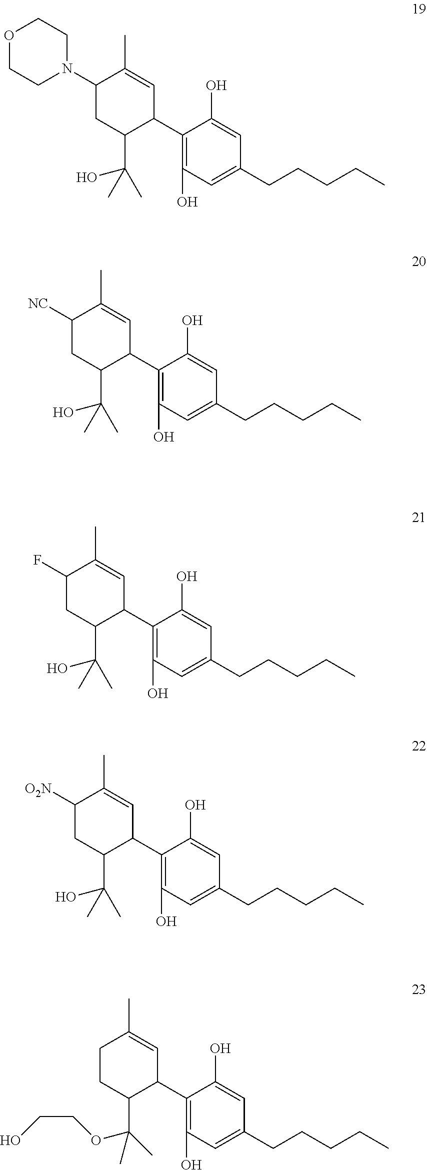 [SMILES: CCCCCc1cc(O)c(C2C=C(C)C(N3CCOCC3)CC2C(C)(C)O)c(O)c1 ; CCCCCc1cc(O)c(C2C=C(C)C(C#N)CC2C(C)(C)O)c(O)c1 ; CCCCCc1cc(O)c(C2C=C(C)C(F)CC2C(C)(C)O)c(O)c1 ; CCCCCc1cc(O)c(C2C=C(C)C([N+](=O)[O-])CC2C(C)(C)O)c(O)c1 ; CCCCCc1cc(O)c(C2C=C(C)CCC2C(C)(C)OCCO)c(O)c1] 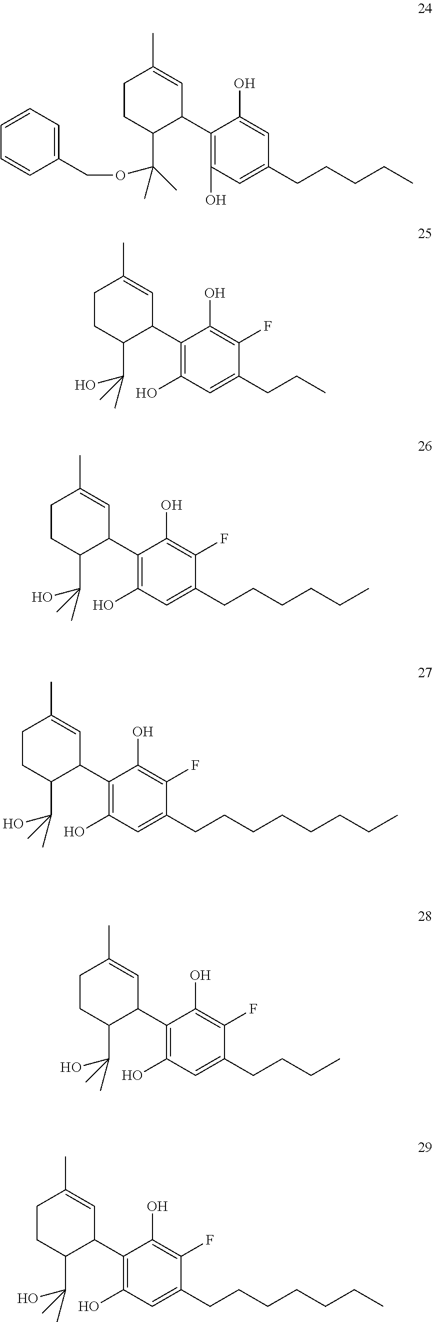 [SMILES: CCCCCc1cc(O)c(C2C=C(C)CCC2C(C)(C)OCc2ccccc2)c(O)c1 ; CCCc1cc(O)c(C2C=C(C)CCC2C(C)(C)O)c(O)c1F ; CCCCCCc1cc(O)c(C2C=C(C)CCC2C(C)(C)O)c(O)c1F ; CCCCCCCCc1cc(O)c(C2C=C(C)CCC2C(C)(C)O)c(O)c1F ; CCCCc1cc(O)c(C2C=C(C)CCC2C(C)(C)O)c(O)c1F ; CCCCCCCc1cc(O)c(C2C=C(C)CCC2C(C)(C)O)c(O)c1F] 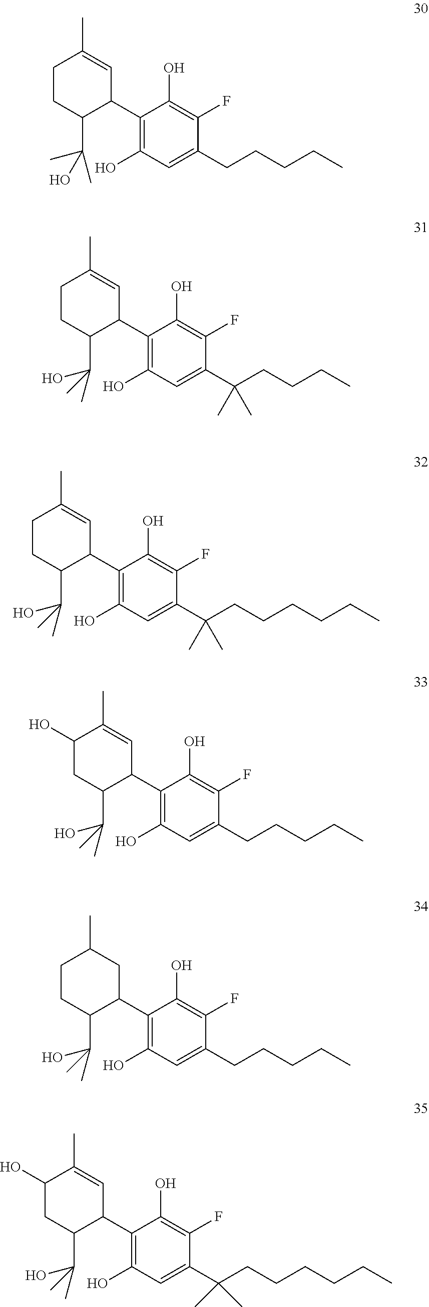 [SMILES: CCCCCc1cc(O)c(C2C=C(C)CCC2C(C)(C)O)c(O)c1F ; CCCCC(C)(C)c1cc(O)c(C2C=C(C)CCC2C(C)(C)O)c(O)c1F ; CCCCCCC(C)(C)c1cc(O)c(C2C=C(C)CCC2C(C)(C)O)c(O)c1F ; CCCCCc1cc(O)c(C2C=C(C)C(O)CC2C(C)(C)O)c(O)c1F ; CCCCCc1cc(O)c(C2CC(C)CCC2C(C)(C)O)c(O)c1F ; CCCCCCC(C)(C)c1cc(O)c(C2C=C(C)C(O)CC2C(C)(C)O)c(O)c1F] 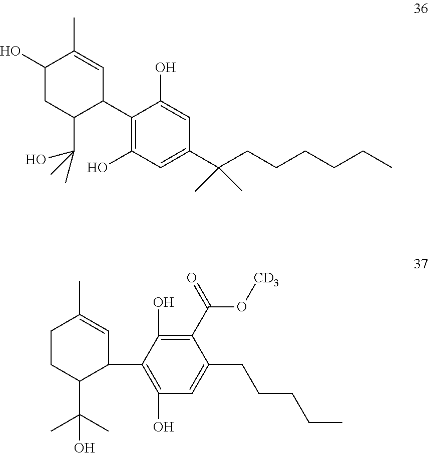 [SMILES: CCCCCCC(C)(C)c1cc(O)c(C2C=C(C)C(O)CC2C(C)(C)O)c(O)c1 ; [2H]C([2H])([2H])OC(=O)c1c(CCCCC)cc(O)c(C2C=C(C)CCC2C(C)(C)O)c1O]
including salts, hydrates, solvates, polymorphs, optical isomers, geometrical isomers, enantiomers, diastereomers, and mixtures thereof.

57. A pharmaceutical composition comprising a therapeutically effective amount of at least one compound of claim 49 and a pharmaceutically acceptable carrier or excipient.

58. The pharmaceutical composition of claim 57, wherein the pharmaceutically acceptable carrier or excipient comprises at least one of a binder, a filler, a diluent, a surfactant or emulsifier, a glidant or lubricant, a buffering or pH adjusting agent, a tonicity enhancing agent, a wetting agent, a chelating agent, a preservative, an antioxidant, a flavoring agent, a colorant, and a mixture or combination thereof.

59. The pharmaceutical composition of claim 57 in a form selected from the group consisting of tablet, pill, capsule, pellets, granules, powder, a wafer, coated or uncoated beads, lozenge, sachet, cachet, elixir, an osmotic pump, a depot system, an iontophoretic system, a patch, suspension, dispersion, emulsion, solution, syrup, aerosol, oil, ointment, suppository, a gel, and a cream.

60. The pharmaceutical composition of claim 57 formulated for administration via a route selected from the group consisting of: oral, topical, transdermal, intra-arterial, intranasal, intraperitoneal, intramuscular, subcutaneous, intravenous, and intra-alveolar.

61. A method of treating a disease or disorder characterized by overproduction of a steroid in a subject in need thereof, the method comprising administering to the subject a pharmaceutical composition comprising a therapeutically effective amount of at least one compound according to claim 49 and a pharmaceutically acceptable carrier or excipient.

62. The method of claim 61, wherein the disease or disorder is selected from the group consisting of: neurodegenerative disorders, metabolic disorders, psychiatric disorders, and trauma-related disorders.

63. A method of treating a proliferative disease or disorder in a subject in need thereof, the method comprising administering to the subject a pharmaceutical composition comprising a therapeutically effective amount of at least one compound according to claim 49 and a pharmaceutically acceptable carrier or excipient.

64. The method of claim 63, wherein the proliferative disease or disorder is cancer.

65. The method of claim 64, wherein the cancer is selected from the group consisting of: head and neck cancer, sarcoma, multiple myeloma, ovarian cancer, breast cancer, bladder cancer, kidney cancer, stomach cancer, hematopoietic cancers, lymphoma, leukemia, lung carcinoma, melanoma, glioblastoma, hepatocarcinoma, prostate cancer, pancreatic cancer, and colon cancer.

66. The method of claim 64, wherein the cancer is selected from the group consisting of: T cell acute lymphoblastic leukemia (T-ALL), chronic lymphocytic leukemia (CLL), melanoma, cholangiocarcinoma (CCC), colorectal cancer, lung adenocarcinoma, glioblastoma, renal cell carcinoma, ovarian cancer, prostate cancer, breast cancer, pancreatic ductal adenocarcinoma (PDAC), cervical cancer, head and neck squamous cell carcinomas (HNSCC), hepatocellular carcinoma (HCC), medulloblastoma, B cell acute lymphoblastic leukemia (B-ALL), acute myeloid leukemia (AML), small cell lung carcinoma (SCLC), non-small-cell lung carcinoma (NSCLC), lung squamous cell carcinoma (SqCC), cutaneous squamous cell carcinoma (SqCC), chronic myelomonocytic leukemia (CMML), cutaneous T-cell lymphoma (CTCL) including mycosis fungoides and Sezary syndrome, intestinal and diffuse-type gastric cancer, pilocytic astrocytoma (PA), choroid plexus tumor, laryngeal squamous cell carcinoma (LSCC), gallbladder carcinoma, Kaposi's sarcoma, B cell malignancy, T cell lymphoma, pancreatic cancer, nasopharyngeal carcinoma, squamous cell carcinoma, prostatic adenocarcinoma, infantile myofibromatosis (IM), lateral meningocele syndrome, and desmoid tumor.

67. The method of claim 64, wherein treating cancer comprises modulating the expression of a NOTCH protein.

68. The method of claim 64, wherein treating cancer comprises modulating an abnormal expression of a NOTCH protein which is encoded by a NOTCH gene comprising at least one mutation.
